(19) United States
(12) Patent Application Publication
(10) Pub. No.: US 2026/0290542 A1
(43) Pub. Date: Sep. 24, 2026

(54) SYSTEM

(71) Applicant: SoftBank Group Corp., Tokyo (JP)

(72) Inventor: Kazumi ISHIWATARI, Tokyo (JP)

(73) Assignee: SoftBank Group Corp., Tokyo (JP)

(21) Appl. No.: 19/560,176

(22) Filed: Mar. 9, 2026

(30) Foreign Application Priority Data
Mar. 19, 2025 (JP) 2025-044552

(51) Int. Cl. G16H 20/60; G06N 3/0475; G06Q 30/0201; G06Q 30/0207

(52) U.S. Cl. CPC G16H 20/60; G06N 3/0475; G06Q 30/0206; G06Q 30/0239

(57) ABSTRACT

A system includes a processor that is configured to collect, from a sensor device, information relating to food ingredients of a user and a health condition of the user, process the collected information by using a data analysis device to analyze a consumption tendency of the food ingredients of the user and the health condition of the user, and generate a prompt sentence that instructs a generative AI model to generate a menu based on an analysis result obtained by the processing.

### CROSS-REFERENCE TO RELATED APPLICATION

This application is based on and claims priority under 35 USC 119 from Japanese Patent Application No. 2025-044552 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional menu planning technologies insufficiently integrate detailed user-specific food ingredient consumption tendencies and health conditions into the process of generating daily or long-term meal plans. In many cases, menu proposals are created based on static user preferences or simple nutritional guidelines, without accurate, sensor-based acquisition of the user's actual ingredient usage or dynamic health status. As a result, the generated menus may not properly reflect the user's real consumption behavior, current health condition, or temporal changes in these factors. Furthermore, existing systems do not effectively leverage generative AI models in a structured manner through prompt sentences that are optimally tailored to analysis results derived from sensor data and data analysis devices. Consequently, it is difficult to generate menus that are both highly personalized to the user's health and ingredient consumption tendencies and easily adaptable over time. Additionally, there is a further problem in that the optimization of menus based on the user's purchase history and coupon information is not sufficiently integrated into an automated pipeline, leading to missed opportunities for cost savings and economical nutrition management. Therefore, there is a need for a system and technique that can (i) collect information related to the user's food ingredients and health condition from sensor devices, (ii) analyze such information to understand the user's consumption tendencies and health status, (iii) generate appropriate prompt sentences for a generative AI model based on such analysis, and (iv) optimize the generated menus using purchase history and coupon information, thereby providing menus that are both health-conscious and cost-effective.

### SUMMARY

In order to solve the above-described problems, according to one aspect of the present invention, there is provided a system comprising a processor, wherein the processor is configured to collect, from a sensor device, information relating to food ingredients of a user and a health condition of the user. The processor is further configured to process the collected information by using a data analysis device to analyze a consumption tendency of the food ingredients of the user and the health condition of the user. Based on an analysis result obtained by such processing, the processor is configured to generate a prompt sentence that instructs a generative AI model to generate a menu. In one embodiment, the processor is configured to input the prompt sentence into the generative AI model and cause the generative AI model to generate a menu suitable for the user, thereby enabling the generation of menus that reflect the user's actual ingredient consumption tendencies and current health status in a dynamic and flexible manner. In another embodiment, the processor is configured to optimize the menu based on a purchase history of the user and coupon information, and to transmit the optimized menu to a terminal of the user. By integrating sensor-based data collection, data analysis of ingredient consumption and health status, prompt generation for a generative AI model, and cost optimization using purchase history and coupon information, the system can provide menus that are personalized in terms of health and ingredient usage while also improving economic efficiency for the user.

The term “system” refers to an apparatus or combination of hardware and software components including at least one processor and optionally including memory, communication interfaces, sensor devices, data analysis devices, and user terminals, configured to execute the functions described in the claims.

The term “processor” refers to any hardware component or set of components capable of executing instructions, including but not limited to a central processing unit (CPU), a graphics processing unit (GPU), a digital signal processor (DSP), or a microcontroller, and may be implemented as a single physical device or as multiple distributed devices operating cooperatively.

The term “sensor device” refers to any device or combination of devices configured to detect or measure information relating to food ingredients of a user and/or a health condition of the user, such as a refrigerator sensor, kitchen scale, barcode scanner, wearable health tracker, biosensor, or environmental sensor, and to output such information in a form processable by the system.

The term “information relating to food ingredients of a user” refers to data that indicate the presence, quantity, type, usage, or consumption pattern of food items associated with the user, including but not limited to inventory status, purchase records, portion sizes, and timestamps of use.

The term “health condition of the user” refers to physical or physiological status information of the user, including but not limited to body weight, body mass index (BMI), blood pressure, heart rate, blood glucose level, nutritional intake, or other medically relevant indicators.

The term “data analysis device” refers to a hardware and/or software component, which may be integrated with or separate from the processor, that is configured to execute statistical analysis, machine learning, rule-based processing, or other computational methods on collected information to derive analysis results such as consumption tendencies and health status evaluations.

The term “consumption tendency of the food ingredients of the user” refers to a pattern or trend in the user's use or intake of specific food ingredients over time, including frequency, quantity, timing, and preference characteristics inferred from sensor data or historical records.

The term “generate a prompt sentence” refers to the act of creating a text or structured natural language instruction that is adapted to be input to a generative AI model and that specifies conditions, constraints, goals, or context for menu generation based on the analysis result.

The term “prompt sentence” refers to a set of textual or structured instructions, queries, or descriptions, expressed in natural or formal language, that are provided as input to a generative AI model in order to cause the generative AI model to produce an output corresponding to a desired menu.

The term “generative AI model” refers to a machine learning model, such as a large language model or other generative model trained on data including culinary, nutritional, or general textual information, that is configured to generate new content, including menu proposals, in response to input prompts.

The term “menu” refers to a set of one or more meal proposals, recipes, or food item combinations for one or more time periods, including associated information such as ingredients, preparation methods, nutritional values, or portion sizes, intended for consumption by the user.

The term “menu suitable for the user” refers to a menu that is generated in consideration of at least one of the user's health condition, ingredient consumption tendencies, preferences, restrictions, or lifestyle, such that the menu is personalized to the user.

The term “purchase history of the user” refers to historical data representing food-related purchases made by or for the user, including at least purchase dates, purchased items, quantities, stores or vendors, and prices.

The term “coupon information” refers to data indicating discounts, promotional offers, or other economic incentives applicable to food items or related services available to the user, including validity periods, applicable products or categories, and discount amounts or rates.

The term “optimize the menu” refers to modifying or selecting the menu in such a way as to improve one or more evaluation criteria, including but not limited to cost, nutritional balance, alignment with health goals, or efficient use of available coupons and purchase history.

The term “terminal of the user” refers to any user-operated device capable of receiving data from the system and presenting information to the user, such as a smartphone, tablet, personal computer, smart display, or other network-connected user interface device.

The term “transmit the optimized menu” refers to sending data representing the optimized menu from the system, via a communication interface, to the terminal of the user so that the user can view or otherwise interact with the optimized menu.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

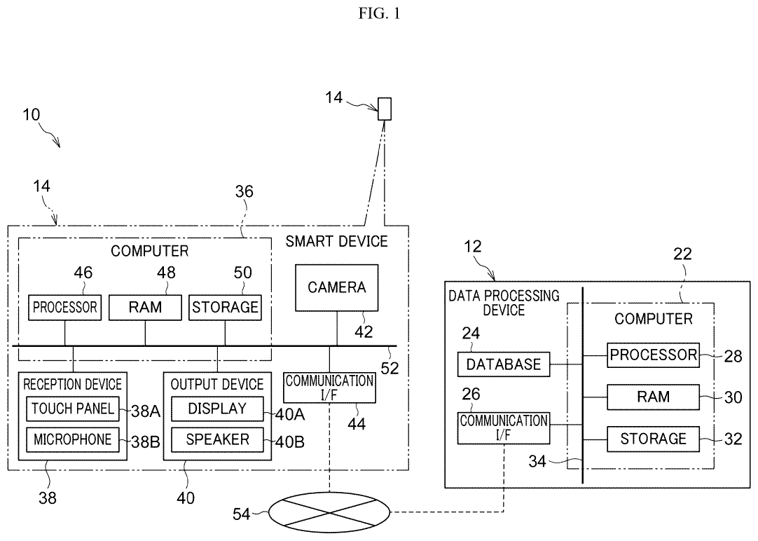

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

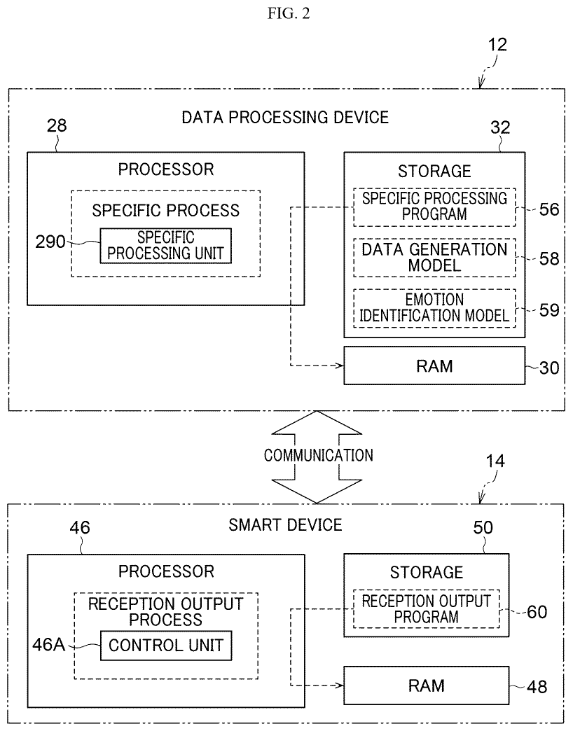

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

In conventional computer-implemented menu recommendation systems, several technical limitations arise from fragmented processing of user interaction data. First, message data received via communication applications is typically handled as unstructured text, and conventional servers often rely on manual rule-based parsing or fixed graphical user interface flows. As a result, the server cannot reliably extract user intent, time ranges, categories, and transaction-related information from natural language messages, which leads to low recall and precision in downstream data access. This degrades the efficiency of data processing pipelines and increases processor load due to repeated parsing and error handling.

Second, conventional systems generally separate the acquisition and storage of transaction data, such as purchase history and coupon information, from the natural language interaction channel. For example, purchase records may be entered via dedicated forms, while natural language queries are processed in a different subsystem. This fragmentation prevents the server from maintaining a consistent, user-centric data model, and forces the processor to perform redundant data conversions and cross-system synchronization. Consequently, response latency increases, and the overall throughput of the information processing apparatus is reduced.

Third, when generative AI models are used, conventional systems typically pass only the user's raw message to the model, without constructing a prompt sentence that systematically embeds machine-readable, structured data retrieved from a database. This underutilizes the capabilities of the generative AI model and causes unstable or inaccurate responses. In addition, ad hoc prompt construction forces the processor to perform repetitive formatting operations for each request, leading to inefficient use of computational resources.

Fourth, existing systems rarely integrate automatic extraction of transactional entities (such as item names, quantities, prices, and transaction dates and times) from free-form messages into a unified storage and retrieval architecture. Without such integration, the server cannot treat communication messages as a reliable source for updating purchase histories and coupon usage data. This limits the ability of the system to self-update from user interactions and requires separate ingestion channels, which complicates the software architecture and increases maintenance overhead.

Therefore, there is a need for an improved computer-implemented system and server architecture that: (i) automatically acquires and analyzes natural language messages from a communication application, (ii) generates structured data representing user intent and transaction parameters in a consistent format, (iii) stores and retrieves user-specific purchase history data and coupon data via a relational database under unified control, and (iv) constructs and supplies optimized prompt sentences to a generative AI model so that the model can generate accurate, context-aware response messages, including optimized menus and associated explanations, with reduced processing overhead and improved response quality. Such an architecture should improve the internal functioning of the server by reducing redundant parsing, optimizing database access patterns, and enabling more efficient use of generative AI resources.

The specific processing by the specific processing unit 290 of the data processing device 12 in Example 1 is realized by the following means.

The present invention provides a server comprising a processor configured to automatically acquire, from an application having a communication processing function, message data in a natural language input by a user; perform, on text information included in the message data, a text analysis process using a natural language processing program, the text analysis process including morphological analysis, part-of-speech tagging, phrase segmentation, semantic analysis, and entity extraction, and generate structured data including parameters indicating a user intent, a time range, a category, and transaction-related information; store, based on the structured data, a data group including purchase history data of the user and coupon data in an information storage device, or acquire the data group from the information storage device, by using a relational database management component, and manage the data group in association with each user; select or aggregate, based on the user intent obtained by the text analysis process and the structured data, the purchase history data and the coupon data acquired from the relational database management component, and generate machine-readable information including a result of the selection or aggregation; generate, based on the machine-readable information and the message data in the natural language from the user, a prompt sentence to be input to a generative AI model, input the prompt sentence to the generative AI model, and cause the generative AI model to generate a response message in the natural language including menu information and an explanatory text corresponding to the menu information; and transmit the response message in the natural language to a terminal of the user as response data for the application having the communication processing function. This enables an improvement in computer functionality in that the server internally transforms unstructured natural language messages into structured data optimized for database access and generative AI interaction, thereby reducing redundant parsing operations, streamlining storage and retrieval of user-specific transactional records, and generating context-aware, menu-related response messages with lower processing latency and higher accuracy.

The term “application having a communication processing function” refers to a software component executed on a terminal or a server that is configured to transmit and receive message data between a user and a server over a communication network, for example through text-based or multimedia-based messaging.

The term “message data in a natural language” refers to electronically stored or transmitted data that includes text expressed in a human language, such as sentences, phrases, or words input by a user for the purpose of conveying an instruction, inquiry, or information.

The term “text information included in the message data” refers to a portion of the message data that consists of character strings or symbols representing natural language content, excluding non-textual elements such as images or binary attachments.

The term “natural language processing program” refers to a software module or set of software modules executed by a processor to analyze natural language text, including functions for linguistic analysis, entity recognition, and semantic interpretation.

The term “text analysis process” refers to a sequence of computational operations applied to text information, including at least one of morphological analysis, part-of-speech tagging, phrase segmentation, semantic analysis, and entity extraction, to generate structured representations of the text.

The term “morphological analysis” refers to a computational operation that segments a text into minimal linguistic units such as morphemes or tokens and determines base forms or inflectional attributes of the units.

The term “part-of-speech tagging” refers to a computational operation that assigns grammatical categories, such as noun, verb, adjective, or adverb, to individual tokens in the text.

The term “phrase segmentation” refers to a computational operation that splits text into meaningful word groups or phrases based on syntactic or statistical criteria.

The term “semantic analysis” refers to a computational operation that infers meanings, roles, or relationships among words and phrases in text to determine the intent or context of the user's message.

The term “entity extraction” refers to a computational operation that identifies and labels specific types of information in text, such as item names, quantities, prices, dates, times, categories, or user identifiers.

The term “structured data” refers to data represented in a predefined format, such as key-value pairs, records, or objects, that encode parameters including user intent, time range, category, and transaction-related information, suitable for storage in and retrieval from a database.

The term “user intent” refers to an inferred purpose or desired operation indicated by the user's message, such as a request to register purchase information, a request to inquire coupon information, or a request to obtain menu information.

The term “time range” refers to a temporal parameter represented by one or more time points or periods, such as a specific date, a set of dates, a week, or another duration, derived from expressions contained in the message data.

The term “category” refers to a classification parameter representing a type or group of items, services, or information, such as a product category, a cuisine type, or a discount type, identified from the message data or associated records.

The term “transaction-related information” refers to information associated with a commercial or consumption event, including at least one of an item name, a quantity, a price, a transaction date, and a transaction time.

The term “data group” refers to a logical set of related data elements, including at least purchase history data and coupon data, associated with a user and managed collectively by the server.

The term “purchase history data” refers to data representing one or more past transactions of a user, including at least identifiers of purchased items, quantities, prices, and transaction dates and times.

The term “coupon data” refers to data representing discount or benefit information, including identifiers of coupons, conditions of use, applicable categories, and validity periods.

The term “information storage device” refers to a physical or virtual storage medium, such as a hard disk drive, solid-state drive, or network-attached storage, configured to store data groups under control of the server.

The term “relational database management component” refers to a software component or set of software components that manages structured data according to a relational data model, executes queries and update operations, and maintains data integrity in the information storage device.

The term “machine-readable information” refers to data formatted in a structure interpretable by a program, such as structured text, records, or serialized objects, and including a result of selection or aggregation of purchase history data and coupon data.

The term “generative AI model” refers to a machine learning model, such as a large language model, configured to generate natural language text or other content based on an input sequence including a prompt sentence and optional structured data.

The term “prompt sentence” refers to a sequence of text, optionally including structured information, constructed for input to a generative AI model to condition or guide the content and style of a generated response.

The term “response message in the natural language” refers to text generated by the generative AI model or by the server, expressed in a human language, and intended to be presented to the user as an answer or explanation.

The term “menu information” refers to information defining one or more proposed meal compositions or food item combinations, optionally associated with nutritional, economic, or preference-related attributes.

The term “explanatory text corresponding to the menu information” refers to natural language text that describes or explains the menu information, including reasons for selection, usage instructions, or related recommendations.

The term “terminal of the user” refers to an information processing device operated by the user, such as a smartphone, tablet, or personal computer, configured to execute the application having a communication processing function and to send and receive message data.

The term “registration request for purchase history” refers to message data in a natural language that instructs the server to store new transaction-related information as purchase history data.

The term “inquiry request for coupon information” refers to message data in a natural language that instructs the server to retrieve and present coupon data that satisfy one or more conditions, such as a time range or a category.

The term “valid period” refers to a time interval during which a coupon represented by coupon data is available for use, as defined by a start date and an end date or equivalent temporal constraints.

The term “optimize the menu information” refers to a computational operation that adjusts or re-ranks menu information based on purchase history data, coupon data, or other user-related parameters, in order to increase suitability, efficiency, or benefit for the user.

Various embodiments of the invention will now be described. The embodiments are illustrative examples and are not intended to limit the scope of the claims. The same or similar functional components may be implemented by different hardware or software configurations, as will be apparent to a person skilled in the art.

### A. Overall Configuration

A server executes a program that acquires natural language message data from a communication application, analyzes the message data by using a natural language processing pipeline, stores and retrieves structured purchase history data and coupon data in a relational database, and generates prompt sentences for a generative AI model in order to obtain response messages including menu information and explanatory text.

The server is implemented by one or more information processing devices, each including at least a processor, a main memory, a non-volatile storage device, and a network interface. In one embodiment, the server includes a general-purpose CPU, such as an x86-compatible processor, a main memory implemented by a DRAM module, and a storage device implemented by a solid-state drive. The server executes an operating system such as a general-purpose server operating system, and executes an application server program and a database management program. The server connects to a network, such as the Internet, through the network interface.

A terminal is implemented by a user device, such as a smartphone, a tablet, or a personal computer. The terminal includes at least a processor, a memory, a display device, an input device (such as a touchscreen or keyboard), and a wireless or wired communication interface. The terminal executes an application having a communication processing function, such as a chat application or messaging interface, which transmits and receives message data to and from the server via a communication network.

A user operates the terminal, inputs message data in a natural language into the communication application, and views response messages presented by the communication application. The user does not need to understand the internal implementation of the server; the user interacts with the system through natural language alone.

### B. Software Modules on the Server

The server executes multiple software modules that cooperate to realize the claimed functions. In one embodiment, the server executes:
(1) a communication interface module configured to receive and transmit message data via HTTP or HTTPS protocols;
(2) a natural language preprocessing module configured to normalize and tokenize text information included in the message data;
(3) a natural language understanding module configured to infer user intent, time ranges, categories, and transaction-related information;
(4) a database access module configured to store and retrieve structured data in a relational database;
(5) a prompt generation module configured to construct prompt sentences for a generative AI model based on structured data and message data;
(6) a generative AI interface module configured to transmit prompt sentences to the generative AI model and receive generated text; and
(7) a response formatting module configured to assemble a response message in a natural language and transmit the response message to the terminal.

In one concrete implementation, the server executes an application framework, such as a Python-based web framework, as the main application program. The server uses a natural language processing library, such as a tokenization and parsing toolkit, and a linguistic analysis framework, to implement the preprocessing and understanding modules. The server uses a relational database management system, such as a general-purpose relational database engine, as the database access module. The server may use an external or internal generative AI model, which is typically implemented as a transformer-based neural network, and accessed through an API.

### C. Natural Language Analysis and Structured Data Representation

The server applies a concrete sequence of natural language processing operations to each received message. The server first performs text normalization, including conversion to a unified character encoding, cancellation of control characters, and canonicalization (for example, uniform handling of full-width and half-width characters in East Asian languages). The server then performs tokenization, which segments the text into tokens such as words, punctuation marks, or sub-word units. In one embodiment, the server uses a rule-based tokenizer combined with a statistical model trained on a corpus of conversational text.

The server performs part-of-speech tagging by applying a sequence labeling model, such as a bidirectional recurrent neural network with a conditional random field layer or an embedded tagging model within a transformer encoder. The part-of-speech tags are used to distinguish content words (such as nouns and verbs) from functional words. The server then performs phrase segmentation and dependency parsing, for example by applying a transition-based parsing algorithm or a graph-based parsing algorithm implemented in the natural language processing library. These analyses allow the server to identify relations such as “object of purchase” or “temporal modifier.”

The server performs entity extraction by using a named entity recognition model. In one embodiment, the server uses a neural network that takes token embeddings as input, where each token embedding is a combination of word embeddings and character-level embeddings. The entity recognition model is trained offline on annotated text containing entity labels such as ITEM_NAME, QUANTITY, PRICE, DATE, TIME, CATEGORY, and LOCATION. The server loads the trained parameters at runtime and applies the model to inference only; no training is required during normal operation.

The server then generates structured data as a set of key-value pairs or a record object. For example, when the user inputs “Please show my purchase history for the last 7 days,” the server generates structured data such as:
intent: GET_PURCHASE_HISTORY
time_range: LAST_7 DAYS
category: (none)
transaction_related_information: (none)

When the user sends a receipt-like text, such as “I bought 2 bottles of milk for 5.00 on January 25,” the server identifies “2” as QUANTITY, “bottles of milk” as ITEM_NAME, “5.00” as PRICE, and “January 25” as DATE, and stores these as elements of transaction-related information.

This structured representation enables the server to perform deterministic database operations without re-parsing the original free-form text. As a result, the server avoids repeated, expensive natural language processing steps for the same logical operation, improving processing speed and reducing processor load.

### D. Database Schema and Data Management

The server uses a relational database management component to manage at least purchase history data and coupon data. The server defines a database schema that includes, for example, a user table, a purchase table, and a coupon table. The purchase table may include fields such as: user_id, item_name, quantity, price, transaction_datetime, and category. The coupon table may include fields such as: coupon_id, description, category, valid_from, valid_to, and condition_text.

The server stores the structured data generated from natural language messages into these tables. For example, when the server extracts transaction-related information for a purchase from a message, the server creates a new row in the purchase table. When the server retrieves coupon data, the server filters rows in the coupon table based on time_range and category parameters included in the structured data.

By using a relational database, the server benefits from optimized query execution plans, indexing, and transactional integrity. This technical arrangement improves data access performance and consistency compared to ad hoc file-based storage or in-memory structures. The server can use indexes on user_id and transaction_datetime to accelerate queries that are constrained by time ranges and user identities. Thus, the specific combination of structured data generation and relational database operations provides a measurable performance improvement.

### E. Prompt Sentence Generation and Generative AI Model

The server generates a prompt sentence as a textual input to a generative AI model. The generative AI model, in one embodiment, is a transformer-based language model that includes multiple layers of self-attention and feed-forward neural networks. The model is trained offline on a large text corpus that includes conversational data and domain-specific examples related to food, purchases, and coupons.

During training, the generative AI model minimizes a loss function, such as cross-entropy between the predicted token distribution and the ground truth tokens. The training process updates the model parameters by gradient descent, e.g., using Adam or a similar optimizer. The training data may be augmented by paraphrasing or back-translation to increase robustness to varied phrasings. After training, the model is deployed to a serving environment and operates in inference mode only.

The server does not merely pass raw user messages to the generative AI model. Instead, the server constructs a prompt sentence that includes both the user's natural language request and the structured machine-readable information retrieved from the database. For example, when the user requests coupon information, the server may generate a prompt sentence such as:
“The user requested: ‘Tell me this week's coupons.’
The following coupons are available this week:
1) Coupon ID C001: 10% off all groceries, valid from January 27 to February 2.
2) Coupon ID C002: Buy 1 get 1 free on coffee, valid from January 29 to February 5.
Please explain these coupons in simple language and mention the validity period of each coupon.”
When the user requests purchase history, the server may generate a prompt sentence such as:
“The user requested: ‘Please show my purchase history for the last 7 days.’
The following purchases were recorded for this user in the last 7 days:
1) Milk, purchased on January 23, price 2.50.
2) Bread, purchased on January 25, price 1.80.
Please summarize this purchase history in one or two concise sentences.”

By embedding structured information into the prompt sentence in a controlled, deterministic format, the server reduces ambiguity for the generative AI model. This results in more stable and accurate outputs, improves the reproducibility of responses, and reduces the need for post-processing and error correction. The combination of database-driven content selection and structured prompt generation constitutes a specific technical mechanism that differs from simply forwarding free-form user text to a generic language model.

### F. Technical Improvement Over Conventional Systems

The server improves computer technology in multiple ways. First, the server introduces a data flow in which unstructured natural language messages are converted into structured data only once at ingestion, and all subsequent operations (database queries, prompt construction, and response generation) operate primarily on structured data. This reduces repeated natural language parsing for similar queries, thereby reducing CPU time and average response latency.

Second, the server uses a specific relational schema and indexing strategy aligned with the extracted parameters (user_intent, time_range, category). As a result, the server can perform targeted SQL queries that directly correspond to the user's abstract request, instead of scanning large text logs. This organizational structure improves cache locality, reduces I/O operations, and permits the database optimizer to generate efficient query plans.

Third, the server uses a distinct prompt generation module that merges structured data with the user's natural language request. This ensures that the generative AI model receives a condensed, relevant context, reducing the number of tokens in the input sequence. Because transformer-based models have computational complexity proportional to the square of the sequence length, reducing the input length directly lowers computation cost and inference latency.

Fourth, the server implements control logic that selects or aggregates data before any generative AI processing occurs. This pre-filtering reduces the number of records that need to be summarized by the generative AI model, making the generation step more focused and efficient. In contrast, conventional systems might ask a generative model to operate on a raw log of messages or records, which is computationally expensive and prone to errors.

Furthermore, the server enforces non-trivial rules in its natural language understanding and prompt generation. For example, the server may impose a rule that purchase entries older than a certain threshold are aggregated into a single summary line before being provided to the generative AI model. The server may also normalize coupon conditions into a standardized phrase set (e.g., “percentage discount,” “buy X get Y”) before inserting them into the prompt. These heuristics and transformation rules are designed to reduce semantic variability and thus increase the accuracy of downstream generation.

### G. Generative AI Model Structure and Usage

The generative AI model, in one embodiment, includes an embedding layer, multiple transformer blocks, and an output projection layer. Each transformer block includes a multi-head self-attention mechanism and a position-wise feed-forward network. The model receives a prompt sentence encoded as a sequence of token IDs. The model computes attention weights between tokens and produces contextual embeddings, which are then used to predict the most likely next token at each position.

The server may set model parameters such as temperature, top-k or top-p sampling thresholds, and maximum output length in order to control the variability and length of the generated text. For example, the server may use a low temperature when generating a concise summary of purchase history, in order to reduce randomness and increase determinism. The server may use a higher temperature for generating menu suggestions to allow more variety.

The server may maintain a vocabulary of domain-specific tokens, such as “low-sodium,” “high-protein,” or “budget-friendly,” which are used during training or fine-tuning of the generative AI model. During inference, the server embeds these tokens in the prompt sentence to steer the model toward domain-appropriate wording.

### H. Alternative Embodiments

In an alternative embodiment, the server implements the natural language processing modules as microservices deployed in containers, and communicates between modules through a message queue. The core operations remain the same: the server generates structured data from natural language text, stores and retrieves data via a relational database, and constructs prompt sentences based on structured data.

In another embodiment, the generative AI model is hosted locally on the same physical server, utilizing a graphics processing unit (GPU) for acceleration. The server transfers the prompt sentence to the local model through shared memory or an internal API. This configuration reduces network latency compared to an external API call and allows more direct control over model parameters and resource allocation.

In yet another embodiment, the server may employ different neural architectures for entity extraction, such as convolutional neural networks or hybrid transformer-recurrent networks, as long as the output is structured data with the required parameters. The selection of model architecture may be tuned based on the expected language, domain, and hardware constraints.

In some embodiments, the server may also perform on-line adaptation of certain parameters, such as user-specific preferences or frequently used categories, stored as additional fields in the database. The server may then include such parameters in the structured data and in the prompt sentence, leading to more personalized yet efficiently generated responses.

### I. Use of the System by the User and Terminal

The user interacts with the system by sending natural language requests through the communication application on the terminal. For example, the user may send:
“Please show my purchase history for the last 7 days.”
“Tell me this week's coupons.”
“Suggest a dinner menu using my recent purchases and available coupons.”

The terminal transmits these requests to the server. The server processes the requests as described above, generates a response message such as:
“In the last 7 days, you purchased Milk on January 23 for 2.50 and Bread on January 25 for 1.80.”
“Here are this week's coupons: 10% off all groceries until February 2, and buy 1 get 1 free on coffee until February 5.”
“Based on your recent purchases and current coupons, a suitable dinner menu is grilled chicken with a side salad and bread. This menu uses items you already bought and allows you to apply the 10% grocery coupon.”

The terminal displays these response messages in the communication application interface. The user may then send follow-up requests, which the server processes in the same manner.

Through these embodiments, the server, terminal, and user cooperate to realize a system in which unstructured natural language communication is efficiently converted into structured database operations and generative responses, thereby improving the internal operation of the computer system beyond mere automation of human tasks.

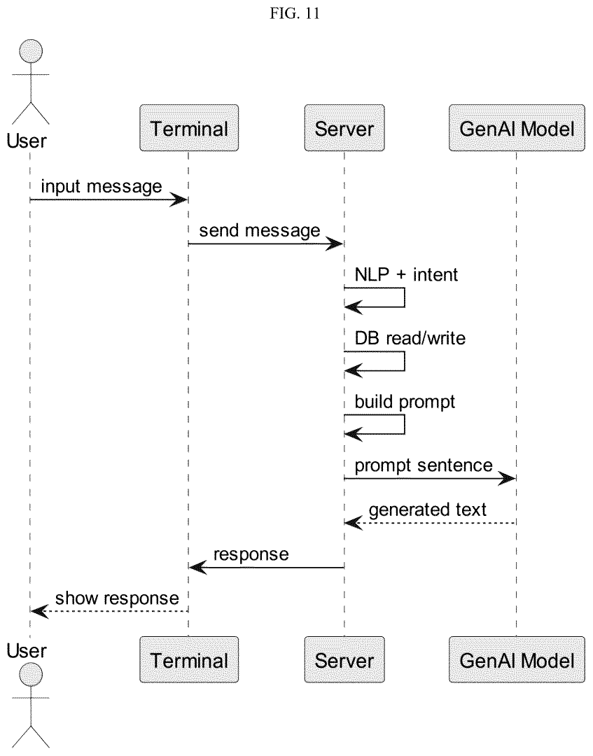

The following describes the processing flow using FIG. 11.

### Step 1:

The user inputs a natural language message into the communication application on the terminal.

The terminal receives the text string typed or spoken by the user, such as “Please show my purchase history for the last 7 days” or “Tell me this week's coupons.”
Input: raw natural language text from the user and associated metadata (user ID, timestamp, application ID).
Output: a structured message object on the terminal containing the text and metadata.

The terminal encapsulates this object into a request payload and transmits it to the server over a network using a communication protocol such as HTTPS.

### Step 2:

The server receives the request payload from the terminal through a communication interface module.

The server parses the incoming HTTP request, validates the payload format, and extracts the message text and metadata fields (user ID, timestamp, application ID).
Input: HTTP request containing a serialized message object from the terminal.
Output: in-memory representation of the message (for example, a record or dictionary) with fields for text, user ID, and timestamp.

The server then forwards this representation to a natural language preprocessing module.

### Step 3:

The server normalizes the text information included in the message data.

The server converts the text to a standard character encoding, removes control characters, normalizes whitespace, and optionally standardizes number formats and date expressions.
Input: raw message text extracted in Step 2.
Output: normalized text string suitable for linguistic analysis.

The server stores the normalized text together with the original text for traceability, so that subsequent modules can rely on a clean, consistent input.

### Step 4:

The server tokenizes and linguistically analyzes the normalized text using a natural language processing program.

The server segments the text into tokens (words, punctuation, or sub-word units), assigns part-of-speech tags, and performs syntactic parsing and phrase segmentation. The server may use a trained sequence labeling model or parser for this operation.
Input: normalized text from Step 3.
Output: a token sequence with associated annotations (tokens, part-of-speech tags, syntactic dependencies, phrase boundaries).

The server generates an intermediate data structure that represents the message at the token level for use in entity extraction and intent recognition.

### Step 5:

The server performs entity extraction to identify transaction-related information and other key elements.

The server applies an entity recognition model or rule set to detect entities such as item names, quantities, prices, dates, times, and categories, and labels the corresponding token spans.
Input: annotated token sequence from Step 4.
Output: a list of entities with types and values (for example, ITEM_NAME=“milk”, QUANTITY=“2”, PRICE=“5.00”, DATE=“January 25”).

The server aggregates these entities into a structured representation that can directly map to database fields.

### Step 6:

The server determines the user intent and extracts control parameters.

The server applies an intent classification model or rule-based logic that analyzes the token sequence and entities to decide whether the message is, for example, a request to register purchase data, an inquiry for purchase history, or an inquiry for coupon information.
Input: entity list and annotated token sequence from Steps 4 and 5.
Output: an intent label (e.g., GET_PURCHASE_HISTORY, GET_COUPONS, REGISTER PURCHASE) and control parameters such as time_range and category.

The server constructs structured data containing at least user_intent, time_range, category, and transaction-related information.

### Step 7:

The server branches processing based on the detected intent.

If the intent corresponds to registration of purchase history, the server prepares to insert or update records in the database. If the intent corresponds to an inquiry, the server prepares to construct database queries using the extracted parameters.
Input: structured data with user_intent and parameters from Step 6.
Output: a control decision indicating either a write operation (insert/update) or a read operation (select) against the database, along with the corresponding database command template.

The serv er forwards the decision and structured data to the database access module.

### Step 8:

The server performs database write operations when the message includes new transaction-related information.

The server maps entities such as item_name, quantity, price, and transaction_datetime into columns of the purchase history table and constructs an INSERT or UPDATE statement. The server may also normalize categories and link them to a category table.
Input: structured transaction-related information and user ID from Step 7.
Output: new or updated rows in the database tables representing purchase history or related records.

The server executes the SQL commands via the relational database management component and commits the transaction to ensure persistence.

### Step 9:

The server performs database read operations when the message is an inquiry for purchase history or coupon information.

The server converts time_range and category parameters into concrete query conditions, such as date intervals and category filters, and constructs SQL SELECT statements.
Input: structured data with user_intent, time_range, and category from Step 7.
Output: a set of records retrieved from the purchase history table and/or coupon table that satisfy the given conditions.

The server retrieves these records, converts them into an internal list of structured objects, and passes them to the prompt generation module.

### Step 10:

The server aggregates and formats the retrieved data into machine-readable information.

The server may group purchase records by date, item, or category, and may filter or sort records to emphasize recent or relevant data. For coupon data, the server may filter coupons to those within the valid period and relevant categories.
Input: raw database result sets from Step 9.
Output: a condensed, ordered list or summary of purchase history and coupon records represented as structured objects (for example, logical lists of “purchase entries” and “coupon entries”).

The server includes metadata such as total counts or aggregated amounts to be used later in generation of human-readable responses.

### Step 11:

The server generates a prompt sentence for a generative AI model based on the machine-readable information and the original user request.

The server constructs a text block that includes the user's initial request followed by a structured description of the retrieved data in natural language, formatted in a consistent pattern. For example, the server outputs lines like “1) Milk, purchased on January 23, price 2.50.”
Input: user's natural language message from Step 2 and structured summary objects from Step 10.
Output: a prompt sentence (or multi-line prompt text) that concisely expresses the user's request and the data to be summarized or explained.

The server passes this prompt sentence to the generative AI interface module.

### Step 12:

The server submits the prompt sentence to the generative AI model and obtains a generated response.

The server transmits the prompt sentence to the generative AI model, which may be hosted locally or accessed through an external API, and sets parameters such as temperature and maximum output length.
Input: prompt sentence from Step 11 and model control parameters (temperature, max tokens, etc.).
Output: a generated natural language response that includes menu information, coupon explanations, or purchase history summaries, depending on the prompt content.

The server receives the generated text from the generative AI model and stores it as a candidate response.

### Step 13:

The server post-processes the generated response message.

The server checks the generated text for length limits, removes extraneous markers or artifacts, and may enforce formatting rules such as line breaks and bullet points. The server may also merge the generated content with fixed template text, such as standard headers or footers.
Input: raw generated text from Step 12.
Output: a cleaned, formatted natural language response message suitable for display on the terminal.

The server ensures that the message is coherent and aligned with the data used in the prompt.

### Step 14:

The server packages and transmits the response message to the terminal.

The server creates a response payload containing the formatted natural language text, and may include additional structured fields such as message type, timestamp, and identifiers for referenced items or coupons.
Input: formatted response message from Step 13 and user metadata (user ID, session ID).
Output: an HTTP or equivalent response sent to the terminal, carrying the natural language response message as part of the payload.

The server logs the request-response pair for monitoring and potential model evaluation.

### Step 15:

The terminal receives and presents the response message to the user.

The terminal parses the received payload, extracts the natural language text, and displays it in the communication application's user interface. The terminal may render line breaks, bullet points, and basic layout as indicated by the formatting rules.
Input: response payload from the server received over the network.
Output: visual presentation of the response message on the display of the terminal.

The user reads the response and may initiate a further interaction by sending another natural language message, which causes the processing flow to repeat from Step 1.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional recommendation systems for shopping and meal planning typically rely on isolated data sources and simple rule-based logic. For example, some systems only use purchase history stored in a database, while other systems only use coupon information or static user profiles. Such systems are not able to effectively integrate heterogeneous data, including transaction record information obtained from communication applications, discount and advertisement information tied to specific sales points, real-time user location information, and dynamically evolving user interaction history. As a result, these systems often generate recommendations that are either irrelevant to the user's current context or redundant with respect to the user's past behavior.

Furthermore, conventional systems that attempt to use machine learning models or generative AI models often treat each model independently. Machine learning models may be used to predict products of interest, and generative AI models may be used to generate natural language text; however, there is no unified, machine-readable mechanism for encoding the full context—such as ranked candidate meal plans, candidate items, location-constrained promotions, and optimized movement routes—into a single, structured prompt sentence. This lack of integrated orchestration leads to inefficient use of computational resources, inconsistent outputs across models, and difficulty in scaling or updating the system behavior as new data arrives.

In addition, many existing systems do not provide a closed feedback loop in which user operation history and newly acquired transaction records are systematically fed back into both the analytical processing and the prompt generation logic. Without such a feedback loop, the system cannot efficiently update the user's feature representation, cannot refine the calculation of candidate items and meal plans, and cannot adapt the content and structure of prompt sentences over time. This results in degraded recommendation quality, reduced user engagement, and suboptimal utilization of discount information and movement route guidance.

From a computer technology standpoint, there is therefore a need for a system architecture and processing method that: (i) automatically acquires and normalizes diverse information assets from communication applications and external sensors into structured data; (ii) combines statistical analysis and machine learning to generate user-specific feature values and ranked candidate meal plans and items; (iii) systematically converts heterogeneous structured data into unified prompt sentences for generative AI models; and (iv) incorporates user feedback and new transaction records into an iterative improvement cycle. Such a system should improve the efficiency, scalability, and accuracy of computer-implemented recommendation processing and provide technically enhanced orchestration between analytical components and generative AI components.

The specific processing by the specific processing unit 290 of the data processing device 12 in Application Example 1 is realized by the following means.

The present invention provides a server comprising a processor configured to acquire, via a communication interface and an external connection mechanism, information assets including transaction record information, discount information, and advertisement information from a communication application for communication between users, automatically convert the acquired information assets into structured data, and store the structured data in a storage device; to receive current location information of a user from a location acquisition mechanism, identify at least one sales point within a predetermined range based on the current location information, and extract, from the storage device, discount information and advertisement information associated with the at least one sales point; to input the transaction record information and purchase history information of the user into an analysis processing mechanism, perform statistical processing and machine learning processing to generate feature values representing a purchase tendency of the user, calculate candidate items and candidate meal plans based on the feature values, and rank the candidate items and the candidate meal plans according to an evaluation index; to compile context information including the ranked candidate items, the ranked candidate meal plans, the current location information, the associated discount information and advertisement information, and movement route information calculated within or between the at least one sales point, convert a plurality of types of structured data in the context information into text format, and generate a unified prompt sentence by embedding the converted text as an integrated instruction statement; to input the unified prompt sentence into a generative AI model operating on a generation processing device, cause the generative AI model to generate natural language text including a recommendation message for the user according to the context information, associate the ranked candidate items and the ranked candidate meal plans with the generated natural language text, and transmit the natural language text, the association, and the movement route information to an information processing terminal of the user; and to receive operation history information of the user and new transaction record information transmitted from the information processing terminal of the user, update the purchase history information and the feature values based on the received information, and iteratively improve both calculation processing of the candidate items and the candidate meal plans and generation processing of subsequent prompt sentences. This enables the server to technically improve recommendation processing by efficiently orchestrating multi-source data acquisition, machine learning-based analysis, and generative AI-based natural language generation in a closed feedback loop, thereby enhancing the precision, context relevance, and computational efficiency of computer-implemented shopping and meal planning assistance.

The term “processor” refers to one or more hardware-based processing units, such as a central processing unit or a graphics processing unit, and associated control logic configured to execute instructions and perform the described operations.

The term “communication interface” refers to a hardware and software interface configured to transmit and receive data between the server and external devices or services, including network interfaces for wired or wireless communication.

The term “external connection mechanism” refers to a combination of hardware and software components configured to connect the server to external information sources, such as application programming interfaces provided by communication applications or external data services.

The term “information assets” refers to digital data items acquired from external sources, including but not limited to transaction record information, discount information, and advertisement information.

The term “transaction record information” refers to digital data representing past commercial transactions, including at least purchase dates, item identifiers, quantities, prices, and store identifiers.

The term “discount information” refers to digital data describing price reductions or preferential conditions for goods or services, including at least discount rates, target items, applicable locations, and validity periods.

The term “advertisement information” refers to digital data describing promotional content related to goods or services, including at least product descriptions, campaign details, and associated sales points.

The term “communication application” refers to a software application that enables message exchange or communication between users over a network, and that is capable of providing information assets to the server.

The term “structured data” refers to data formatted in a predefined schema, such as a table or key-value representation, that allows programmatic access to individual fields including transaction record information, discount information, and advertisement information.

The term “storage device” refers to one or more hardware storage units, such as non-volatile memory or magnetic storage, configured to store structured data and other information used by the system.

The term “current location information” refers to digital data representing a geographic position of a user, including at least coordinates, and obtained from a location acquisition mechanism.

The term “location acquisition mechanism” refers to hardware and software components configured to detect or estimate a geographic position of a user, including positioning systems and related APIs.

The term “sales point” refers to a physical or virtual location at which goods or services are offered to users, including at least retail facilities and service provision points.

The term “purchase history information” refers to accumulated transaction record information of a user over time, representing the user's past purchases across one or more sales points.

The term “analysis processing mechanism” refers to software components, and optionally dedicated hardware, configured to perform analytical operations on data, including statistical processing and machine learning processing.

The term “statistical processing” refers to computational operations that calculate statistical measures, such as frequencies, averages, and distributions, from purchase history information or related data.

The term “machine learning processing” refers to computational operations that apply machine learning models to input data in order to infer patterns or predictions, such as user purchase tendencies.

The term “feature values” refers to numerical or categorical values derived from raw data, representing characteristics of a user's behavior or status, and used as input to analytical or predictive models.

The term “purchase tendency” refers to a pattern or preference in a user's purchasing behavior, inferred from feature values derived from purchase history information and related data.

The term “candidate items” refers to goods or services selected by the analysis processing mechanism as potential recommendations for a user based on feature values and other information.

The term “candidate meal plans” refers to meal configurations, such as sets of dishes or menus, generated by the analysis processing mechanism as potential proposals for a user based on feature values and available items.

The term “context information” refers to a collection of data elements that characterize the current recommendation situation, including at least candidate items, candidate meal plans, current location information, discount information, advertisement information, and movement route information.

The term “prompt sentence” refers to a text-based instruction or query that encodes context information and is input to a generative AI model to control generation of natural language text.

The term “generation processing device” refers to hardware and software components configured to execute a generative AI model and produce natural language text or other outputs based on a prompt sentence.

The term “generative AI model” refers to a machine learning model, such as a language model, configured to generate natural language text or other content in response to a text-based input including a prompt sentence.

The term “natural language text” refers to text expressed in a human language, generated by the generative AI model, and intended to be understandable by a user.

The term “recommendation message” refers to natural language text that includes suggestions of items, meal plans, or movement routes tailored to a specific user based on context information.

The term “information processing terminal” refers to a user-operated device, such as a mobile terminal or computing terminal, configured to communicate with the server and present information to the user.

The term “movement route” refers to a sequence of locations or waypoints within at least one sales point or between multiple sales points, calculated to guide a user in acquiring candidate items corresponding to candidate meal plans.

The term “movement route information” refers to digital data describing a movement route, including at least an ordered list of waypoints, segment connections, or travel instructions.

The term “operation history information” refers to digital data representing user actions performed on an information processing terminal, including at least selections, confirmations, and dismissals related to presented recommendations.

The term “new transaction record information” refers to transaction record information obtained after a previous analysis or update, representing newly occurring purchases or interactions.

The term “evaluation index” refers to one or more numerical or categorical indicators computed for candidate items or candidate meal plans, used to determine ranking or priority in recommendations.

In one embodiment, a server, a terminal, and a user cooperate in a networked environment to implement the invention. The server comprises at least one processor, a main memory, and a non-volatile storage device such as a solid-state drive. The server further comprises a network interface providing packet-based communication over a data network. The terminal comprises a processor, a memory, a display unit, an input unit, a wireless communication interface, and a location acquisition mechanism such as a global positioning system receiver and associated operating system location APIs. The user operates the terminal in physical proximity to one or more sales points such as retail facilities.

The server executes software components implemented as a set of cooperating modules. In one embodiment, the server executes a backend application realized using a framework such as a web application framework running on an operating system. The server uses a database management system such as a structured query database or a document-oriented database, and an in-memory data structure library. The server further uses a data analysis library such as a tabular data analysis library, a machine learning library such as a general-purpose machine learning toolkit, and a deep learning framework such as a tensor-based computation library. The server uses a generative AI model, for example a transformer-based language model deployed either as a remote service or as a locally hosted neural network.

The server acquires information assets from one or more communication applications operated by the user on separate devices. The server uses a communication interface and an external connection mechanism to invoke an application programming interface provided by the communication application. The server receives message data including transaction record information, discount information, and advertisement information in a structured or semi-structured representation such as a JavaScript object notation format. The server stores incoming payloads temporarily in main memory, decomposes each payload into message header fields and content fields, and persists them to the storage device using a schema defined in the database management system.

The server converts unstructured or semi-structured information assets into structured data. The server uses a parsing module that applies regular expression matching, dictionary-based tokenization, and optional optical character recognition to extract item identifiers, quantities, prices, timestamps, store identifiers, discount rates, and validity periods. The server uses a hierarchical data structure in which each transaction record is represented as a record with a primary key and a list of item sub-records, and in which each discount or advertisement entry is represented as a record associated with one or more store identifiers and product categories. By normalizing heterogeneous message formats into a unified data schema, the server enables subsequent analytical modules to operate on consistent data structures, thereby reducing parsing overhead in later processing and improving overall processing speed.

The terminal acquires current location information of the user by invoking an operating system location acquisition mechanism. The terminal periodically obtains latitude, longitude, accuracy, and timestamp values from the location acquisition mechanism and maintains these values in a local cache. The terminal uses a wireless communication interface to transmit the location data to the server. The terminal attaches a user identifier or session identifier to the location data to enable association with stored purchase history in the server.

The server receives current location information and updates a user profile data structure stored in the storage device. The server uses a geospatial indexing mechanism, such as geohash-based indexing or a spatial extension of the database, to map the received coordinates to one or more nearby sales points. The server maintains a sales point table that includes each sales point's location coordinates, logical identifier, and optional layout metadata such as aisle identifiers and shelf positions. By using geospatial indexing, the server performs proximity searches using optimized tree or hash structures rather than linear scans, which reduces query latency and improves scalability as the number of sales points increases.

The server analyzes purchase history information and transaction record information using a dedicated analysis processing mechanism. The server retrieves historical transaction records for a particular user and loads them into a tabular in-memory representation. The server derives feature values from the raw purchase data by grouping purchase events by item category, computing frequencies over configurable time windows, calculating recency values such as a number of days since last purchase per category, and computing monetary metrics such as average expenditure per category. The server further constructs temporal features such as preferred shopping days of the week and typical times of day for purchases.

In one embodiment, the server uses a machine learning model implemented in a machine learning library to map the feature values to predicted future purchase probabilities. The server represents each user as a feature vector including elements such as normalized purchase frequencies, log-transformed expenditure values, and categorical encodings of preferred stores and product groups. The server trains a supervised model, such as a gradient boosting decision tree ensemble or a random forest classifier, on historical data where labels indicate whether particular items were purchased within a target time window. The server uses a loss function such as cross-entropy or logistic loss and an optimization algorithm implemented in the machine learning library to fit model parameters. The server evaluates model quality using performance metrics such as precision, recall, and area under a receiver operating characteristic curve, and periodically retrains the model using newly collected data.

The server generates candidate items and candidate meal plans based on predicted purchase probabilities and recipe relationships. The server uses a recipe database that associates each meal plan with a set of required ingredients and metadata such as preparation time and nutritional content. The server maps predicted high-probability products to ingredient categories and identifies meal plans that can be partially or fully assembled with predicted items and items already purchased in recent history. The server computes an evaluation index for each candidate meal plan and each candidate item by combining predicted purchase probability, economic savings due to applicable discounts, and coverage of required ingredients. The server may use a weighted linear combination of normalized sub-scores or a learned ranking function. This ranking operation is executed by the analysis processing mechanism and produces an ordered list of candidate items and candidate meal plans.

The server generates context information by combining ranked candidate items, ranked candidate meal plans, current location information, active discount information, and movement route information. The server structures this context information in an intermediate representation that explicitly identifies item identifiers, store identifiers, discount parameters, and route segments. The server calculates movement routes within or between sales points by representing the sales point layout as a graph where nodes correspond to aisle positions or store entrances and edges correspond to walkable paths. The server uses a graph search algorithm such as Dijkstra's algorithm or a heuristic shortest path algorithm to minimize estimated walking distance or time given the set of locations containing the candidate items. The server outputs a sequence of waypoints as movement route information.

The server constructs a prompt sentence for a generative AI model using the context information. The server converts structured fields into human-readable textual fragments according to predefined templates. For example, the server converts a discount record into a phrase such as “there is a 10% discount on vegetables at the nearest supermarket” and converts a ranked meal plan into a phrase such as “a recommended dinner menu is pasta with tomato sauce and salad.” The server concatenates these fragments and instruction phrases into a unified prompt sentence.

In one example, the server constructs the following prompt sentence:
“The user frequently buys milk, bread, and eggs on weekday evenings. The user is currently at a supermarket where there is a 10% discount on vegetables and a special price on fresh milk. The recommended products are: 1) fresh milk, 2) lettuce, 3) tomatoes, 4) bread rolls. Please generate a friendly, concise recommendation message that suggests a simple dinner menu and explains why these items are suitable.”

In another example, the server constructs the following prompt sentence:
“The user is currently inside a supermarket. According to the user's recent purchase history, the user often buys yogurt, cereal, and bananas for breakfast. Today, there is a coupon for 20% off yogurt and 10% off bananas. The recommended items are: 1) Greek yogurt, 2) whole-grain cereal, 3) bananas. Please generate a short, friendly recommendation message (about 2-3 sentences) that suggests a healthy breakfast using these items and clearly mentions that the user can save money by using today's coupons.”

The server inputs the prompt sentence into a generative AI model. In one embodiment, the generative AI model is a transformer-based language model with a plurality of encoder-decoder layers or decoder-only layers. The model comprises an embedding layer that converts tokens of the prompt sentence into numerical vectors, a stack of self-attention and feed-forward layers that compute contextualized representations, and an output layer that predicts probabilities over vocabulary tokens. The server controls hyperparameters such as maximum sequence length, sampling temperature, and top-k or nucleus sampling thresholds to balance determinism and diversity of generated text.

The server generates natural language text by performing autoregressive decoding, repeatedly selecting next tokens according to the model's output probabilities and assembling them into sentences. The server may impose constraints such as maximum length and avoidance of prohibited terms. Because the server encodes ranked candidate items, ranked candidate meal plans, and movement route information into the prompt sentence in a structured, machine-controlled manner, the generative AI model is guided to produce text that explicitly mentions relevant items, meal plans, and navigation hints. This approach improves the alignment between the model's output and the system's analytical results compared to naïve prompting based only on free-form summaries.

The server associates generated natural language text with corresponding structured data. The server maintains an association record linking each generated recommendation message to internal identifiers of candidate items, candidate meal plans, and movement route segments referenced in the underlying context information. This linkage enables the terminal to present interactive user interfaces in which the user can tap on textual recommendations to reveal structured details such as item prices, discount expiration dates, and aisle locations.

The terminal receives recommendation messages, structured item data, and movement route information from the server. The terminal displays the generated natural language text to the user using the display unit and simultaneously renders graphical elements such as item cards and maps. The terminal uses stored movement route information to draw a route on a store map or to list stepwise instructions such as “First, go to aisle 2 for pasta, then go to aisle 3 for tomato sauce, then go to aisle 5 for vegetables.” The user views the recommendations and route guidance and moves within the sales point accordingly.

The user performs operations on the terminal, such as selecting recommended items, dismissing particular suggestions, or modifying a suggested meal plan. The terminal records these actions as operation history information and periodically transmits them back to the server. In some embodiments, the user additionally allows new transaction record information to be captured from communication applications or point-of-sale systems and stored by the server. The server merges this incoming data with existing purchase history information and recalculates feature values. Because the feature values and ranking calculations are continuously updated, subsequent prompt sentences incorporate evolving behavioral patterns and discount conditions, which in turn modifies the generative AI model's output.

From a computer technology standpoint, the described architecture and algorithms provide technical improvements over conventional systems. By defining and enforcing specific data structures for transaction records, discount information, advertisement information, and movement routes, the server reduces the need for repetitive parsing in different components and enables efficient indexing and retrieval. By performing feature extraction and machine learning-based ranking prior to prompt construction, the server compresses high-dimensional behavioral data into a smaller set of salient features and ranked candidates. This compression reduces the length of prompt sentences needed to convey relevant context to the generative AI model, which decreases computation time in the model and network transmission size.

The server improves accuracy of recommendations and reduces error compared to manual rule-based systems by using machine learning models with explicitly defined training procedures. The server selects algorithm types, specifies loss functions such as logistic loss, and performs weight updates using gradient-based optimization. The server may employ cross-validation and regularization techniques to prevent overfitting. These practices yield more reliable prediction of user preferences, which directly affects the ranking of candidate items and the structure of context information. The generative AI model, when controlled by well-structured prompt sentences, then produces natural language recommendations that are more contextually appropriate and less redundant.

The server further improves computational efficiency by separating processing into modular stages: acquisition and normalization, analytical modeling, context assembly, prompt generation, and generative text decoding. Each module uses optimized data structures and algorithms suitable for its function. For example, the analysis processing mechanism uses vectorized operations of the tabular data library, which operate on entire columns of data in memory to accelerate computation of statistics and feature vectors. The geospatial module uses indexed spatial queries to avoid full-table scans. The movement route computation uses efficient graph algorithms. These concrete algorithmic choices and data structures result in measurable reductions in processing time per recommendation cycle.

The system also achieves communication load reduction by sending summarized context representations and generated text instead of raw historical data from the server to the terminal. The server performs heavy computation centrally and transmits only the recommended items, meal plans, and compact route information. This division of labor reduces bandwidth requirements on the network and reduces processing load on the terminal.

The server processes information in ways that differ from human manual reasoning. For example, the server computes evaluation indices using multidimensional feature vectors and trained models that combine frequency, recency, monetary, and spatial dimensions, which no human can systematically apply at scale and speed. The server dynamically adjusts prompt sentence content based on real-time model outputs and route calculations, using a rule set that optimizes token usage for the generative AI model and ensures that critical context is retained while redundant details are discarded. This non-conventional, model-driven prompt construction pipeline constitutes more than a mere automation of human decision-making; it represents an engineered method for controlling generative models to operate as integrated components of a recommendation engine.

In alternative embodiments, the server may employ different machine learning algorithms such as neural network-based recommenders with embedding layers for items and users, or may use different generative architectures such as encoder-decoder models. The server may use different feature sets, such as incorporating environmental sensor data indicating store crowding levels, or nutritional goals specified by the user. The server may also vary the movement route optimization algorithm by including constraints such as avoiding congested aisles or minimizing time near refrigeration units, which modifies the cost function of the route calculation.

In another embodiment, the terminal may additionally use short-range communication mechanisms, such as proximity-based beacons, to refine location estimates inside a sales point. The server may incorporate these refined positions into its movement route computation, leading to more accurate waypoint sequences and reduced walking distance. This tighter coupling between physical movement and digital route computation highlights the system's connection to real-world device control and physical navigation.

Because the server uses specific data structures, feature extraction mechanisms, machine learning models, prompt generation logic, and generative model control procedures as described above, the system as a whole provides technical effects such as increased processing speed, improved recommendation accuracy, reduction of network traffic, and decreased computational cost per recommendation. These improvements result from concrete modifications to computer operation rather than from mere business rule automation.

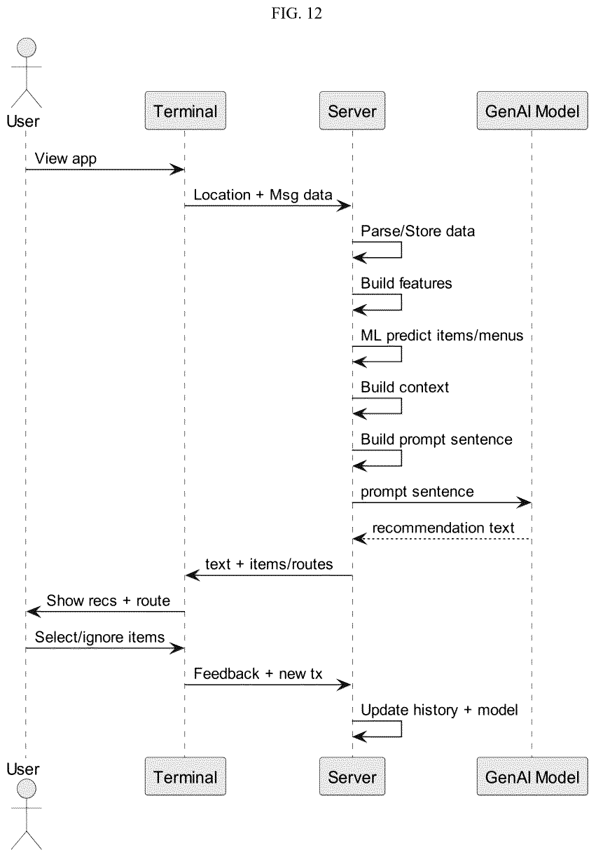

The following describes the processing flow using FIG. 12.

### Step 1:

The server acquires information assets from a communication application.

The server receives, as input, message data from a communication application via an application programming interface, including message text, metadata, and attached files. The server parses the input by separating headers (sender ID, timestamp, message ID) from content (text body, file references), and stores the raw payloads in a temporary buffer in memory. The server then normalizes the data by converting character encodings, removing unsupported control characters, and packaging the cleaned messages into a standard internal format. The output of this step is a set of normalized message objects ready for further extraction of transaction record information, discount information, and advertisement information.

### Step 2:

The server converts message content into structured transaction, discount, and advertisement records.

The server receives, as input, the normalized message objects from Step 1. The server applies rule-based parsing, regular expression matching, and, where necessary, optical character recognition on attached images to identify receipt-like structures (line items, totals), coupon patterns (percentages, conditions), and advertisement segments (promotional phrases, product names). The server maps detected fields into predefined schemas for transaction records, discounts, and advertisements, creating structured records with explicit fields such as item identifiers, quantities, unit prices, discount rates, and validity periods. The output of this step is a set of structured database entries that represent transaction record information, discount information, and advertisement information stored in a storage device.

### Step 3:

The server updates user-specific purchase history and sales point databases.

The server receives, as input, the structured records generated in Step 2. The server associates each transaction record with a user identifier, a sales point identifier, and a timestamp, and merges the new records into existing purchase history tables. The server also updates sales point tables to reflect any new or changed promotional conditions derived from discount and advertisement records. The server performs data aggregation operations such as incrementing per-item purchase counts and updating last-purchase timestamps. The output of this step is an updated set of purchase history information and an updated set of sales point promotion data, both stored in persistent storage.

### Step 4:

The terminal acquires current location information of the user.

The terminal receives, as input, signals from a location acquisition mechanism such as a global positioning system receiver and inertial sensors. The terminal invokes operating system location APIs to compute a current location estimate in terms of latitude, longitude, accuracy, and timestamp. The terminal then formats this data into a location payload that also contains a terminal identifier or session token. The output of this step is a location payload stored in the terminal's memory and prepared for transmission to the server.

### Step 5:

The terminal transmits location information to the server.

The terminal receives, as input, the location payload generated in Step 4. The terminal opens a secure network connection using a wireless communication interface and sends the payload to a server endpoint via a network protocol such as HTTPS. The terminal may compress the payload and add integrity checksums to reduce transmission size and improve reliability. The output of this step is the transmitted location information arriving at the server as a request message.

### Step 6:

The server associates the user location with nearby sales points.

The server receives, as input, the location payload from Step 5. The server extracts the coordinates and user identifier, and queries a geospatial index structure in the database to find sales points whose stored coordinates fall within a predefined radius around the user's current position. The server computes distances using a geodesic or planar distance formula and selects sales points sorted by distance. The output of this step is a list of nearby sales points, each with identifiers, coordinates, and associated metadata.

### Step 7:

The server retrieves active discount and advertisement information for nearby sales points.

The server receives, as input, the list of nearby sales points from Step 6. The server uses sales point identifiers as keys to query promotion tables containing discount information and advertisement information, filtering for records whose validity periods include the current time. The server performs selection and join operations to attach relevant promotions to corresponding sales points. The output of this step is a structured list of active promotions, each linked to a sales point and stored in a context data structure.

### Step 8:

The server computes user feature values from purchase history.

The server receives, as input, updated purchase history information from Step 3 and the user identifier associated with the current session. The server loads the user's historical records into an in-memory tabular data structure and performs aggregation operations such as grouping by item category, summing quantities, and averaging prices. The server calculates feature values such as purchase frequencies over defined time windows, recency measurements, monetary spending scores, and preferred sales point indicators. The server additionally encodes categorical variables as numerical features using techniques such as one-hot encoding. The output of this step is a feature vector representing the user's purchase tendency.

### Step 9:

The server predicts candidate items using a machine learning model.

The server receives, as input, the feature vector produced in Step 8. The server passes this vector through a trained machine learning model implemented in a machine learning library, such as a gradient boosting ensemble or a random forest classifier. The server applies the model's prediction function to compute purchase probability or relevance scores for a predefined catalog of items. The server then sorts items by their scores and selects a subset that exceeds a threshold or that ranks within a top-k range. The output of this step is a ranked list of candidate items predicted to be of interest to the user.

### Step 10:

The server generates candidate meal plans based on predicted items and recipes.

The server receives, as input, the ranked candidate items from Step 9 and a recipe database. The server maps candidate items to ingredient categories and queries the recipe database for meal plans that include these categories as required or optional ingredients. The server evaluates each recipe by checking how many required ingredients are already covered by the candidate items and by items recently purchased in the user's history. The server assigns coverage scores and cost estimates to each recipe and orders them according to a computed evaluation index. The output of this step is a ranked list of candidate meal plans, each with ingredient coverage and estimated cost information.

### Step 11:

The server calculates movement routes for acquiring candidate items.

The server receives, as input, the ranked candidate items from Step 9 and sales point layout data corresponding to nearby sales points identified in Step 6. The server represents the layout as a graph structure, assigns graph nodes to aisles or locations where items are stored, and maps candidate items to specific nodes. The server runs a shortest path or tour optimization algorithm over the graph to find a route that visits nodes associated with the candidate items while minimizing estimated travel distance or time. The server encodes the result as an ordered list of waypoints with associated actions. The output of this step is movement route information linked to candidate items and sales points.

### Step 12:

The server assembles context information for prompt construction.

The server receives, as input, the ranked candidate items from Step 9, the ranked candidate meal plans from Step 10, the active promotions from Step 7, the movement route information from Step 11, and the current location information from Step 6. The server aggregates this input into a context structure, grouping data by user and nearest sales point. The server filters out low-relevance entries based on threshold rules and compresses the context by retaining only the top-ranked items and meal plans. The output of this step is a compact, structured context object that encapsulates all information necessary for generating a prompt sentence.

### Step 13:

The server generates a prompt sentence for a generative AI model.

The server receives, as input, the context object produced in Step 12. The server converts structured fields into natural language fragments using template rules, such as transforming discount records into phrases describing current offers, and transforming ranked candidates into ordered lists of recommended products and meal plans. The server concatenates these fragments with instruction text that specifies the desired style and length of the output. For example, the server may generate the prompt sentence: “The user frequently buys milk, bread, and eggs on weekday evenings. The user is currently at a supermarket where there is a 10% discount on vegetables and a special price on fresh milk. The recommended products are: 1) fresh milk, 2) lettuce, 3) tomatoes, 4) bread rolls. Please generate a friendly, concise recommendation message that suggests a simple dinner menu and explains why these items are suitable.” The output of this step is a single prompt sentence encoded as text.

### Step 14:

The server obtains a recommendation message from a generative AI model.

The server receives, as input, the prompt sentence generated in Step 13. The server forwards this text to a generative AI model, such as a transformer-based language model hosted locally or accessed via a remote service. The server sets decoding parameters such as maximum token count and sampling temperature, and instructs the model to generate a sequence of tokens as a response. The server collects the generated tokens, reconstructs them into sentences, and performs optional post-processing to remove undesired phrases or enforce formatting rules. The output of this step is a natural language recommendation message that references the context encoded in the prompt sentence.

### Step 15:

The server associates structured recommendations with the generated message and sends them to the terminal.

The server receives, as input, the natural language recommendation message from Step 14 and the context object from Step 12. The server creates association records that map textual mentions in the message to internal identifiers of candidate items, candidate meal plans, and movement route segments. The server then constructs a response payload containing the message, the associated structured data, and sales point identifiers. The server transmits this payload to the terminal over a network connection. The output of this step is a combined recommendation package received by the terminal.

### Step 16:

The terminal presents recommendations and routes to the user.

The terminal receives, as input, the recommendation package from Step 15. The terminal parses the payload, extracts the natural language recommendation message, the list of candidate items and meal plans, and the movement route information. The terminal displays the message to the user on the display unit and renders graphical user interface elements such as item cards and route maps, aligning them with the associated text. The terminal may also generate notifications or alerts when the user is near a relevant sales point. The output of this step is a user interface state in which the user can see and interact with recommendations and route guidance.

### Step 17:

The user interacts with recommendations and executes purchases.

The user receives, as input, the displayed recommendation message, item list, and route visualization shown on the terminal. The user reads the content, decides which items to select, and operates the terminal by tapping, scrolling, or confirming suggested meal plans. The user may then move along the indicated route in the physical sales point and add selected items to a shopping basket. The user's actions generate implicit and explicit feedback such as selected items and ignored suggestions. The output of this step is a set of user interaction events and, eventually, new transaction record information representing completed purchases.

### Step 18:

The terminal records user interaction history and transmits feedback to the server.

The terminal receives, as input, low-level user interaction events (touch events, button presses, list selections) and higher-level actions derived from these events, such as “item accepted” or “suggestion dismissed.” The terminal encodes these actions into operation history records that include timestamps, referenced item identifiers, and action types. The terminal sends these records, along with any newly available transaction record information captured through the terminal, to the server over the network. The output of this step is a feedback payload delivered to the server for use in updating models and histories.

### Step 19:

The server updates models and data based on feedback.

The server receives, as input, the feedback payload from Step 18, including operation history information and new transaction record information. The server merges new transaction records into the purchase history data structures, updates counters, and recalculates statistics such as frequencies and recency. The server also updates training datasets for the machine learning model, optionally retraining or incrementally updating model parameters to reflect the latest behavior. The server recalculates feature values for the user and stores them for future sessions. The output of this step is an updated state of purchase history, feature vectors, and optionally updated model parameters, which will be used as input to Steps 8 and 9 in subsequent cycles.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented menu recommendation systems typically rely on static rule sets, simple preference scores, or manually curated recipes. These systems often treat purchase history data and discount information as auxiliary display elements rather than as primary optimization signals. As a result, conventional systems fail to fully exploit large volumes of structured transactional data and pricing data stored in databases when generating menu proposals.

Furthermore, although generative AI models have recently become capable of producing rich natural language recipes and meal plans from free-form prompts, existing systems generally use such models in an ad hoc manner. In many cases, the construction of prompt sentences is hard-coded or manually adjusted, and does not systematically reflect user-specific consumption tendencies, discount usage tendencies, or cost structures. This leads to suboptimal utilization of computing resources, as the generative AI model generates outputs without being constrained or guided by the underlying structured data and without clear machine-readable formatting instructions. Consequently, downstream processing, such as cost calculation and optimization, remains fragile and computationally inefficient.

In typical architectures, processors do not normalize and aggregate purchase history information and discount information in a way that can be robustly linked to AI-generated menu components. Ingredient names generated by a generative AI model frequently deviate from standardized database terminology, which complicates automatic association with transaction records and discount records. This mismatch forces additional manual mapping or heuristic post-processing and prevents accurate, automated calculation of estimated costs and discount effects. As a result, the overall computer system exhibits low efficiency, increased latency, and reduced reliability in generating economically optimized menus at scale.

Additionally, conventional systems do not encode, within the prompt sentence itself, explicit constraint information such as planning period, meal category, nutritional conditions, cooking time conditions, and a required structured output format. Without such constraints, the generative AI model tends to output unstructured or inconsistent text, which requires complex parsing logic and additional error handling. This increases processor workload, memory usage, and network overhead when multiple iterations of prompt refinement and parsing are needed.

Accordingly, there is a need for an improved computer-implemented technique that: (i) systematically aggregates and normalizes purchase history and discount information; (ii) analyzes user-specific tendencies; (iii) generates prompt sentences that explicitly encode these analysis results together with constraint information; (iv) causes a generative AI model to output structured menu candidate information; and (v) automatically associates the AI-generated menu components with standardized ingredient data, transactional data, and discount data in order to compute and optimize economic efficiency. Such a technique should improve the overall performance, scalability, and reliability of the computer system itself, by reducing unnecessary computation, minimizing manual intervention, and enabling more deterministic downstream processing of generative AI outputs.

The specific processing by the specific processing unit 290 of the data processing device 12 in Example 2 is realized by the following means.

The present invention provides a server comprising a processor and an information storage device, the processor being configured to receive request information including user identification information from a user terminal; access the information storage device based on the request information to retrieve purchase history information and price discount information associated with the user; aggregate and normalize the retrieved purchase history information and price discount information to extract frequent item information and applicable discount information and to analyze a food ingredient consumption tendency and a price discount usage tendency of the user; generate, in a natural language, a prompt sentence including at least the frequent item information and the applicable discount information and further specifying constraint information including a menu planning period, a meal category, nutritional conditions, cooking time conditions, and output format conditions; input the generated prompt sentence into a generative AI model and obtain menu candidate information output in a structured format from the generative AI model; normalize ingredient names included in the menu candidate information based on standardized ingredient information stored in the information storage device; associate the normalized ingredient information with the purchase history information and the price discount information to identify ingredients that the user is likely to already possess and ingredients to which a price discount is applicable; calculate, for each menu candidate, an estimated cost and a discount application effect based on the association; evaluate and rank the menu candidates based on economic efficiency and ingredient utilization efficiency; generate optimized menu information according to a result of the evaluation; and transmit the optimized menu information to the user terminal. This enables the computer system to more efficiently utilize structured transactional and discount data in combination with generative AI outputs, to reduce processing complexity and parsing overhead by enforcing structured AI responses, to improve the accuracy and determinism of automatic cost and discount calculations, and to enhance overall system performance and scalability when generating economically optimized, user-specific menu proposals.

The term “processor” refers to a hardware computation unit, such as a central processing unit or a microcontroller, that executes instructions of one or more programs to perform data acquisition, data processing, prompt generation, AI interaction, evaluation, and communication functions in the system.

The term “information storage device” refers to a non-transitory computer-readable storage medium, such as a hard disk drive, a solid-state drive, or a semiconductor memory, that stores purchase history information, price discount information, standardized ingredient information, programs, and intermediate or final data used by the processor.

The term “user terminal” refers to an electronic device operated by a user, such as a smartphone, a tablet, a personal computer, or a similar communication device, that transmits request information to the server and receives and displays optimized menu information.

The term “request information” refers to data transmitted from the user terminal to the server, including at least user identification information and optionally additional condition designation information such as a menu planning period, a meal category, nutritional conditions, or cooking time conditions.

The term “user identification information” refers to information that uniquely or pseudo-uniquely identifies a user within the system, such as a user ID, an account ID, or an authentication token, and is used by the processor to retrieve data associated with the user from the information storage device.

The term “purchase history information” refers to structured data representing past acquisition or purchase events of food ingredients or related items by the user, including, for example, item identifiers, item names, categories, quantities, purchase dates, and prices.

The term “price discount information” refers to structured data representing discount conditions applicable to items or item categories, such as coupon records or promotional discount records, including discount rates, discount types, applicable item categories, validity periods, and other parameters relevant to price reduction.

The term “frequent item information” refers to data derived from aggregation of purchase history information that indicates items or item categories purchased by the user with a frequency or quantity exceeding a predetermined threshold.

The term “applicable discount information” refers to data derived from price discount information that identifies discounts applicable to one or more of the items or item categories related to the user's purchase history or to ingredients used in menu candidates.

The term “food ingredient consumption tendency” refers to a pattern or profile of a user's behavior regarding acquisition and use of food ingredients, inferred from the purchase history information through statistical aggregation or analysis.

The term “price discount usage tendency” refers to a pattern or profile of a user's behavior regarding utilization of discounts, inferred from correlations between purchase history information and price discount information, such as frequency or conditions of discount usage.

The term “condition designation information” refers to constraint or preference information provided by the user, such as desired menu planning period, meal category, nutritional conditions, cooking time conditions, or format preferences, which the processor uses to guide menu generation.

The term “prompt sentence” refers to a sequence of natural language text that encodes, in human-readable form, analysis results, user preferences, constraint information, and format instructions, and that is provided as input to the generative AI model to cause the model to generate menu candidate information.

The term “generative AI model” refers to a machine-learned model, such as a large language model or similar generative model, that is configured to receive a prompt sentence as input and to output generated text including menu candidate information according to the instructions and constraints specified in the prompt sentence.

The term “menu candidate information” refers to information generated by the generative AI model in response to the prompt sentence, including at least proposed menus or dishes, associated ingredients, and cooking procedures, and optionally being structured in a machine-readable format.

The term “structured format” refers to an output format in which elements such as days, dish names, ingredients, and cooking procedures are organized according to predefined fields or a schema, such as a hierarchical or key-value structure, that facilitates programmatic parsing and processing by the processor.

The term “standardized ingredient information” refers to reference data stored in the information storage device that defines canonical names, identifiers, categories, and related attributes for ingredients, and that is used by the processor to normalize ingredient names appearing in purchase history information and menu candidate information.

The term “normalized ingredient information” refers to ingredient data in which ingredient names or identifiers have been converted to standardized forms defined by the standardized ingredient information, enabling consistent association with purchase history information and price discount information.

The term “economic efficiency” refers to a measure used by the processor to evaluate menu candidates based on factors such as estimated total cost, utilization of discounts, and reduction of additional purchases, and may be expressed as a score, ranking, or similar indicator.

The term “ingredient utilization efficiency” refers to a measure used by the processor to evaluate how effectively a menu candidate uses ingredients that the user is likely to already possess, inferred from purchase history information, thereby reducing waste and unnecessary purchases.

The term “optimized menu information” refers to menu information generated by the processor after evaluating and ranking menu candidates based on economic efficiency and ingredient utilization efficiency, and selecting or reordering menu candidates to best satisfy predetermined optimization criteria for the user.

In one embodiment, a server cooperates with a user terminal to generate optimized menu information by combining structured transactional data, discount data, and outputs of a generative AI model. The server includes at least one processor and an information storage device. The processor executes computer programs stored in the information storage device. The information storage device includes a database implemented, for example, using a relational database management system such as a general-purpose SQL database, and a file system or key-value store for storing learned parameters and configuration data of the generative AI model.

The server stores, in the database, purchase history information, price discount information, and standardized ingredient information. The purchase history information is stored in tables having fields including item identifier, item name, category identifier, quantity, unit price, purchase date, and user identifier. The price discount information is stored in tables having fields including discount identifier, discount type, discount rate, applicable category identifier, start date, and expiration date. The standardized ingredient information is stored in tables having fields including ingredient identifier, canonical ingredient name, category identifier, unit, and optionally nutritional attributes.

The server stores, in the information storage device, one or more classifier models and configuration files used to analyze food ingredient consumption tendencies and price discount usage tendencies. For example, the server uses an execution environment based on an interpreted language runtime, such as a Python interpreter, in combination with a numerical computation library such as a general array-processing library and a tabular data-processing library such as a general data frame library. The server uses such libraries to organize purchase history information into data frame structures, each data frame including columns for item identifiers, category identifiers, quantities, dates, and prices.

The server aggregates purchase history information per user by grouping data frame rows by item identifier or category identifier and computing aggregate values such as total quantity, number of purchases, and average unit price. The server normalizes item names by mapping raw item name strings to canonical ingredient names stored in the standardized ingredient information, using lookup tables and, in some cases, string similarity algorithms. This normalization enables the server to represent ingredients in a consistent, machine-readable form, which is essential for subsequent association with generative AI outputs.

The server analyzes food ingredient consumption tendencies by constructing feature vectors for each user. The server converts aggregated counts and frequencies into numerical feature vectors, where each dimension corresponds to a standardized ingredient or category. The server optionally applies dimensionality reduction or clustering algorithms, such as k-means clustering, to identify groups of ingredients frequently purchased together by the user. The server also computes temporal features, such as purchase frequency per weekday or per month, by aggregating purchase history by time windows. The server stores analysis results as structured records containing frequent item information and derived preference scores.

The server analyzes price discount usage tendencies by linking purchase history records and price discount records. The server joins tables on time ranges and applicable categories, and determines, for each purchase event, whether a discount was available and whether it was actually applied. The server then computes features such as the ratio of discounted purchases to total purchases per category and the responsiveness of the user to particular discount types. The server stores these features in an analysis table or as serialized feature vectors. By performing this analysis, the server improves data management and allows efficient reuse of feature vectors in subsequent processing, rather than recomputing complex joins for each request.

The server maintains a generative AI model in the form of a trained neural network. In one embodiment, the generative AI model is implemented as a transformer-based neural network. The transformer architecture includes an embedding layer, multiple stacked self-attention layers, feed-forward layers, and an output layer projecting to a vocabulary space. The server stores the model weights, including weight matrices for attention mechanisms and feed-forward layers, in the information storage device. The server also stores configuration parameters such as the number of layers, the size of hidden representations, attention head counts, and vocabulary size.

The server initially trains the generative AI model on large-scale text corpora containing recipe texts, menu descriptions, and general language data. During training, the server uses a supervised learning algorithm such as stochastic gradient descent with variants like Adam optimization. The server defines a loss function such as cross-entropy loss between predicted token probabilities and ground-truth tokens. The server updates the model weights by backpropagation, computing gradients of the loss with respect to the weights and adjusting the weights according to the optimization algorithm. The server may apply regularization techniques, such as dropout and weight decay, and data augmentation techniques, such as random truncation and permutation of recipe steps, to improve generalization.

The server optionally fine-tunes the generative AI model on domain-specific data such as historical menu plans and user feedback. The server uses reinforcement-style or preference-based learning by encoding feedback signals (for example, menu selections or rejections) as labels, and adjusting model outputs to better match user-accepted menus. The server maintains separate configuration for inference, specifying parameters such as sampling temperature, top-k or top-p sampling thresholds, and maximum output length, to balance creativity and determinism in menu generation.

The server generates a prompt sentence in natural language by combining analysis results and user-provided constraints. The server stores prompt templates as string patterns with placeholders for frequent item information, applicable discount information, planning period, meal category, nutritional requirements, cooking time constraints, and output format instructions. The server fills in these placeholders with concrete values derived from the database and the user's request. This template-based approach ensures that the prompt sentence always includes machine-parsable cues, such as explicit references to structured fields the generative AI model is expected to output.

For example, the server generates a prompt sentence such as:
“The user frequently buys chicken, carrots, and potatoes and currently has a 10% off coupon for vegetables. Please generate a 3-day dinner menu that mainly uses chicken, carrots, and potatoes, takes advantage of the vegetable discount, and keeps each recipe simple (under 30 minutes cooking time). For each day, output the menu name, the list of ingredients, and step-by-step cooking instructions in a clearly structured format.”

In another example, the server generates a prompt sentence such as:
“The user often purchases tofu, leafy greens, and whole grains and has active discounts for plant-based products. Based on this, please propose a 5-day dinner plan suitable for a health-conscious adult, with low-sodium recipes. Include, for each day, a dish name, ingredients, and 4-6 concise cooking steps. Clearly separate the days and label each section.”

The server sends the generated prompt sentence to the generative AI model for inference. The server encodes the prompt sentence into token IDs using a tokenizer corresponding to the transformer vocabulary. The server then feeds the token sequence into the transformer network. The transformer computes, layer by layer, attention scores between tokens and derives contextualized embeddings. The output layer generates probability distributions over possible next tokens. The server selects tokens according to predetermined sampling rules (for example, top-k sampling with a specified k and temperature) to construct the output text. The server limits the number of tokens to control computation time and communication overhead.

The server receives the generated output text from the generative AI model and applies a deterministic parsing method guided by the format instructions included in the prompt sentence. Because the prompt sentence explicitly requests clearly separated days and clearly labeled sections, the server can use pattern matching, section labels, and delimiter-based splitting rather than generic natural language parsing. This reduces processor load and improves reliability of subsequent processing relative to systems that parse arbitrary unstructured text.

The server normalizes ingredient names contained in the generated menu candidate information. The server first performs tokenization of the generated text into candidate ingredient lines. The server then matches each line against the standardized ingredient information using exact matching and approximate string matching (for example, edit distance or n-gram similarity). The server resolves ambiguous cases by considering associated category hints or co-occurrence patterns learned from training data. By using standardized ingredient information, the server enables robust linking between AI-generated text and structured database records. This normalization step is not a mere automation of human editing but a specific algorithmic procedure that reduces error rates in ingredient mapping and improves computational efficiency, because subsequent cost calculations can operate over numeric identifiers instead of free-text strings.

The server associates normalized ingredient information with purchase history information and price discount information. The server executes join operations between normalized ingredient identifiers and the purchase history table to determine whether the user recently purchased the corresponding ingredients. The server estimates stock availability by applying rules such as comparing purchase dates to assumed consumption periods and adjusting by average usage per meal. The server also joins ingredient identifiers with price discount information to determine which discounts apply to each ingredient. The server computes estimated prices by retrieving unit prices from past transactions or from reference price tables and applying discount rates where applicable.

The server calculates an estimated cost and a discount application effect for each menu candidate. The server sums estimated costs of ingredients per dish and applies discounts to determine discounted totals. The server computes metrics such as total cost, total discount amount, proportion of ingredients likely already available, and inferred waste reduction. The server then evaluates and ranks menu candidates using a multi-criteria scoring function that combines economic efficiency and ingredient utilization efficiency. This scoring function is implemented as a numerical algorithm executed by the processor, which may use weighted sums, normalization, and thresholding to select a final subset of menu candidates. The use of such a scoring function provides a predictable, machine-implementable optimization process that improves over naive or ad hoc selection of AI outputs.

The server generates optimized menu information by selecting the highest-ranked menu candidates and formatting them into a structured representation stored, for example, as JSON-like records in the database or in transient memory. The optimized menu information contains, for each day or for each menu item, a menu name, a list of normalized ingredients with associated estimated costs and discount flags, and step-by-step cooking instructions as generated by the generative AI model. The server then transmits this optimized menu information to the user terminal over a network using protocols such as HTTPS. The server may compress the response payload or omit redundant fields to reduce communication load.

The terminal receives the optimized menu information and renders it for display using a graphical user interface framework. The terminal parses the structured data and displays, for example, a day-by-day list of menus, each with a concise description, ingredient list, and visual indicators for ingredients already available or subject to discounts. The terminal may allow the user to view details such as breakdown of estimated cost and discount contributions. The terminal may further enable the user to submit feedback, such as marking dishes as preferred or not desired. The server records this feedback and may incorporate it into subsequent feature vectors used for analyzing tendencies, thereby refining the guidance provided to the generative AI model and improving the overall system performance.

The server achieves technical effects that go beyond mere automation of human planning. By aggregating and normalizing data using specific data structures and libraries, the server reduces the number of required database queries and avoids repeated expensive joins, thereby improving processing speed. By constructing prompt sentences that encode explicit constraints and format instructions, the server causes the generative AI model to produce outputs that are easier to parse, reducing parsing errors and CPU time needed for downstream processing. By normalizing AI-generated ingredient names against standardized ingredient information and linking them to purchase history and discount data, the server enables accurate, automated cost and discount calculations that would be difficult and error-prone for human operators. As a result, the system reduces error rates in menu-cost estimation and discount application and improves the precision of optimization.

The server further improves computational efficiency by offloading linguistic generation to the generative AI model while retaining deterministic control over optimization and evaluation logic. The combination of model-guided generation and structured post-processing yields menus that are both contextually rich and computationally tractable. Because the generative AI model is trained and fine-tuned with explicit loss functions and update rules, the server can systematically adapt model behavior to reduce average response length or to align outputs more closely with the required structure, thus reducing bandwidth consumption and post-processing overhead.

In alternative embodiments, the server may use different model architectures or analysis algorithms. For example, the server may use a recurrent neural network with attention mechanisms instead of a transformer, or may employ gradient-boosted decision trees for analyzing consumption and discount tendencies. The server may also use different feature sets, such as nutritional features or seasonality indicators, and different optimization criteria, such as environmental impact scores. The server may be deployed on a single physical machine or on a distributed cluster, and may use a combination of in-memory caches and persistent storage to balance speed and durability.

In all such embodiments, the server executes a sequence of specialized data-structuring, analysis, prompt construction, generative inference, normalization, association, and evaluation operations that are tightly coupled to the characteristics of the hardware, storage structures, and neural network models. This coupling yields technical improvements in processing speed, scalability, reliability of data association, and accuracy of cost optimization, thereby improving the functioning of the computer system itself rather than merely organizing human business activities.

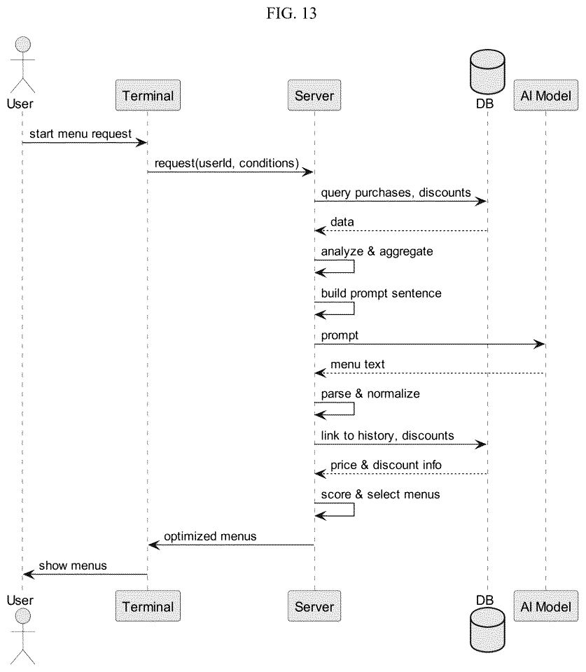

The following describes the processing flow using FIG. 13.

### Step 1:

The user operates the terminal to start a menu recommendation.

The terminal displays an input screen and the user inputs planning conditions such as the number of days, meal category (for example, dinner), dietary preferences, and time constraints.
Input: User operations (touch, click, keyboard input) specifying user identification information (for example, a stored user ID) and planning conditions.

The terminal packages the user ID and planning conditions into a request message in a structured format and sends the request to the server via a network protocol such as HTTPS.
Output: A request message containing the user ID and planning conditions transmitted from the terminal to the server.

### Step 2:

The server receives the request message and extracts the user ID and planning conditions.
Input: The request message from the terminal containing the user ID and planning conditions.

The server parses the message body, validates the user ID, and stores the extracted parameters in working memory as variables or data structures.

The server logs the request metadata (for example, timestamp, user ID, request type) in a log table or file for monitoring and debugging.
Output: In-memory structures holding a validated user ID and associated planning conditions.

### Step 3:

The server retrieves purchase history information and price discount information from the information storage device.
Input: The validated user ID and planning conditions.

The server constructs SQL queries referencing purchase history tables and discount tables in a relational database, filtering by user ID and, optionally, by date range or validity period.

The server executes the SQL queries using a database driver and obtains result sets containing rows of purchase records and discount records.

The server converts the result sets into tabular in-memory structures, such as data frames, for subsequent processing.
Output: Purchase history data and price discount data in tabular in-memory form associated with the user.

### Step 4:

The server aggregates and normalizes the retrieved purchase history data.
Input: The tabular purchase history data.

The server maps raw item names in the purchase records to standardized ingredient identifiers using a lookup table stored in the standardized ingredient information.

The server performs grouping operations by standardized ingredient identifier or by category identifier, computing aggregate values such as total quantity, number of purchase events, and average unit price.

The server computes purchasing frequency metrics, such as purchases per week or per month, using purchase dates and time-window functions.

The server generates a list of frequent items by selecting standardized ingredients whose purchase frequency or quantity exceeds a predetermined threshold.
Output: A data structure containing normalized purchase data and frequent item information for the user.

### Step 5:

The server analyzes food ingredient consumption tendencies and price discount usage tendencies.
Input: Normalized purchase data, frequent item information, and price discount data.

The server joins normalized purchase records with discount records based on applicable categories and time periods to identify purchases made under discount conditions.

The server computes usage ratios for each discount type and category by dividing the number of discounted purchases by the total number of purchases.

The server constructs feature vectors representing, for each user, ingredient preferences and discount usage patterns, where each dimension corresponds to an ingredient, category, or discount type.

The server optionally applies analytical algorithms, such as clustering or regression, to identify patterns such as groups of ingredients frequently purchased together or categories sensitive to discounts
Output: Analysis results including frequent item information, discount usage tendencies, and user-specific feature vectors.

### Step 6:

The server generates a prompt sentence based on the analysis results and the planning conditions.
Input: Analysis results (frequent items, discount tendencies) and planning conditions (for example, planning period, meal category, nutritional constraints, cooking time constraints).

The server selects a prompt template, which is a natural language string pattern with placeholders for items, discounts, and constraints.

The server fills the placeholders with concrete values, such as ingredient names (“chicken”, “carrots”, “potatoes”), discount descriptions (“10% off vegetables”), number of days, and requested recipe simplicity.

The server appends explicit instructions regarding output format, such as requesting separate sections per day, listing dish names, ingredients, and step-by-step instructions.

For example, the server generates a prompt sentence such as:
“The user frequently buys chicken, carrots, and potatoes and currently has a 10% off coupon for vegetables. Please generate a 3-day dinner menu that mainly uses chicken, carrots, and potatoes, takes advantage of the vegetable discount, and keeps each recipe simple (under 30 minutes cooking time). For each day, output the menu name, the list of ingredients, and step-by-step cooking instructions in a clearly structured format.”
Output: A complete natural-language prompt sentence ready to be supplied to the generative AI model.

### Step 7:

The server inputs the generated prompt sentence into the generative AI model and obtains menu candidate information.
Input: The prompt sentence generated in Step 6.

The server tokenizes the prompt sentence into a sequence of token identifiers using a tokenizer associated with the generative AI model.

The server feeds the token sequence into the generative AI model, which processes the tokens through its neural network layers to compute probability distributions for subsequent tokens.

The server generates an output token sequence by sampling tokens from the probability distributions according to predefined parameters (for example, temperature, top-k, or top-p).

The server converts the output token sequence back into a natural-language text string functioning as menu candidate information.
Output: Menu candidate text including multiple proposed menus or dishes, with ingredients and cooking instructions.

### Step 8:

The server parses the menu candidate text to extract structured menu candidate information.
Input: The natural-language menu candidate text from the generative AI model.

The server detects separators and labels in the text (for example, “Day 1”, “Ingredients:”, “Instructions:”) guided by the format instructions in the prompt sentence.

The server splits the text into sections for each day or menu, and further splits each section into fields such as dish name, ingredient list, and sequence of cooking steps.

The server constructs an internal structured representation (for example, objects or records) that maps each day to its corresponding dish name, ingredient list, and steps.
Output: Structured menu candidate information with explicit fields for days, dish names, ingredient lists, and instructions.

### Step 9:

The server normalizes ingredient names in the structured menu candidate information.
Input: Structured menu candidate information and standardized ingredient information stored in the information storage device.

The server iterates over each ingredient name in the menu candidate information and attempts to match it to a canonical ingredient name using exact string comparison and approximate string matching techniques.

The server uses the standardized ingredient table to resolve synonyms and variations (for example, mapping “chicken breast” and “chicken thighs” to a single canonical ingredient identifier when appropriate).

The server stores, for each menu candidate, a list of normalized ingredient identifiers and associated canonical names.
Output: Menu candidate information with ingredients represented by normalized ingredient identifiers and canonical names.

### Step 10:

The server associates normalized ingredient information with purchase history information and price discount information.
Input: Normalized ingredient identifiers for each menu candidate, purchase history data, and price discount data.

The server performs join operations between ingredient identifiers and purchase history records for the user to determine recent purchase events for each ingredient.

The server estimates stock availability by comparing purchase dates to assumed consumption periods and by considering aggregated quantities.

The server also joins ingredient identifiers with price discount records to determine which discounts currently apply to each ingredient.

The server calculates, for each ingredient, estimated unit prices and discounted unit prices, using either historical prices or reference price tables.
Output: Ingredient-level association data, including stock availability estimates, applicable discounts, and estimated prices for each ingredient in each menu candidate.

### Step 11:

The server calculates estimated costs and discount effects for each menu candidate and evaluates menu candidates.
Input: Ingredient-level association data for all menu candidates.

The server sums the estimated costs of all ingredients within a menu candidate to compute a total estimated cost.

The server computes total discount amounts by summing the differences between undiscounted and discounted prices for ingredients to which discounts apply.

The server calculates metrics such as economic efficiency scores based on total cost, discount utilization, and proportion of ingredients likely already available.

The server evaluates and ranks menu candidates using a scoring function that combines these metrics, for example by assigning weights and computing a composite score for each candidate.
Output: Ranked menu candidates with associated cost, discount, and efficiency metrics.

### Step 12:

The server generates optimized menu information and sends it to the terminal.
Input: Ranked menu candidates and efficiency metrics.

The server selects one or more top-ranked menu candidates according to predetermined criteria, such as highest economic efficiency score or user-specified constraints.

The server formats the selected menu candidates into an optimized menu data structure, including, for each day, the dish name, normalized ingredient list with price and discount annotations, and step-by-step instructions.

The server serializes the optimized menu data structure into a response message and transmits it to the terminal over the network using a communication protocol such as HTTPS.
Output: A response message containing optimized menu information delivered from the server to the terminal.

### Step 13:

The terminal receives and displays the optimized menu information, and the user interacts with the proposed menus.
Input: The response message from the server containing optimized menu information.

The terminal parses the response message into internal data structures and updates the user interface to present the menus in a human-readable form, such as a list of days and dishes.

The terminal highlights ingredients that are likely already available and those covered by discounts, based on flags received from the server.

The user reviews the proposed menus, selects dishes, and may provide feedback such as marking menus as preferred or requesting alternatives.
Output: Displayed optimized menus on the terminal and, optionally, user feedback events that can be sent back to the server for future analysis.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented meal recommendation and shopping support systems typically treat recommendation logic as a simple application-level function that maps static user attributes and product data to menu suggestions. Such systems generally (i) operate on fragmented data silos (for example, separate stores for purchases, health metrics, coupons, and location), (ii) invoke recommendation engines or machine learning models with ad-hoc, non-structured input strings, and (iii) do not maintain a closed feedback loop to improve subsequent computation and model invocation. As a result, these systems frequently generate recommendations that are sub-optimal with respect to computational efficiency, data utilization efficiency, and adaptability of the underlying processing pipeline.

In particular, in conventional architectures, the application server often passes raw or only lightly preprocessed user data directly to a generative model or rule-based engine without an intermediate abstraction layer that (a) normalizes heterogeneous data sources into structured data, (b) derives multi-dimensional states such as ingredient consumption tendency, health state, budget state, and emotion state, and (c) programmatically encodes such states into a machine-constructed prompt sentence. This lack of a systematic, processor-enforced pipeline leads to increased server-side processing overhead, redundant model calls, and poor controllability of the generative model's output domain, thereby limiting the scalability and reliability of the computer system.

Further, many existing systems do not natively integrate route computation and economic optimization into the same processing pipeline that generates the recommendations. Purchase route computation and coupon application, if present at all, are typically handled in a loosely coupled post-processing stage. This fragmentation results in redundant queries to external services (for example, map services and promotion providers), non-deterministic behavior across sessions, and additional latency. The computer resources are therefore not used efficiently, and the overall throughput and responsiveness of the system degrade as the number of users and data sources increases.

Moreover, conventional systems rarely manage user feedback and evaluation data as first-class computational inputs that update the logic by which prompts are constructed and by which analytical and optimization steps are parameterized. Without such a learning loop at the system level, the processor continues to execute the same static sequence of operations and prompt construction rules, regardless of long-term user behavior and satisfaction. This makes the system brittle and reduces its ability to converge toward efficient, user-specific configurations of the generative AI model and associated optimization modules.

Accordingly, there is a need for an improved computer-implemented system and server-side processing architecture that (i) unifies heterogeneous user-related information into structured data, (ii) derives and maintains rich user states including consumption tendency, health, budget, position, and emotion, (iii) programmatically generates structured prompt sentences for a generative AI model based on those states and on external provision information, (iv) optimizes the generated plans in conjunction with sales promotion and route computation data, and (v) dynamically updates analysis and prompt-generation conditions based on user feedback. Such a system should improve the technical performance of the computer itself, for example by reducing redundant data transfers and model invocations, controlling the generative model's output space more deterministically, minimizing route-computation overhead, and enabling adaptive tuning of processing parameters over time.

The specific processing by the specific processing unit 290 of the data processing device 12 in Application Example 2 is realized by the following means.

The present invention provides a server comprising a processor configured to acquire, via an information acquisition module, user-related information including ingredient information, health information, preference information, behavior history information, position information, and emotion information from at least one sensing device, user terminal, or external information service; to integrate and organize the acquired user-related information as structured data, and to analyze, via an analysis module, based on the structured data, at least an ingredient consumption tendency of the user, a health state of the user, a budget state of the user, and an emotion state of the user; to generate, via a prompt generation module, a prompt sentence described in a natural language, the prompt sentence specifying, based on a result of the analysis and on external provision information including at least sales promotion information, discount information, and distribution information, at least one of a meal plan or menu plan, a purchase route, and purchase target items to be provided to the user; to convert, via a generation-result acquisition module, the prompt sentence into input data for a generative AI model, to input the input data to the generative AI model, and to extract, from an output result of the generative AI model, at least a candidate of a meal plan or menu plan and necessary ingredient information corresponding to the candidate; to optimize, via an optimization module, the meal plan or menu plan based on the necessary ingredient information and the sales promotion information and the discount information such that the meal plan or menu plan satisfies at least a budget condition and a health condition of the user while achieving cost reduction and burden reduction for the user; to calculate, via a route calculation module, based on the position information and the necessary ingredient information, at least one of available supply locations or provision locations, and, using route information, to compute an optimal purchase route or delivery option; to provide, via a providing module, the meal plan or menu plan, the necessary ingredient information, the purchase route, and the delivery option to the user terminal, to cause display of these on the user terminal, and to execute, based on a selection operation on the user terminal, at least one of order processing or fixation processing of an execution plan; and to update, via a learning module, at least one of analysis conditions used by the analysis module or generation conditions used by the prompt generation module based on evaluation information and feedback information acquired from the user terminal. This enables the computer system to perform an integrated and adaptive processing pipeline that (i) reduces redundant access to heterogeneous data sources by normalizing user-related information into structured data, (ii) constrains and guides the generative AI model through systematically constructed prompt sentences so as to improve determinism and relevance of generated plans, (iii) incorporates promotion and route computation into the same optimized decision process to reduce computational overhead and latency for purchase-route calculation, and (iv) continuously refines analytical and prompt-generation parameters based on user feedback, thereby improving the efficiency, scalability, and responsiveness of the underlying server-side computing resources.

The term “user-related information” refers to information associated with a user, including at least ingredient information, health information, preference information, behavior history information, position information, and emotion information, which is used by the system to analyze and generate plans for the user.

The term “ingredient information” refers to information indicating types, quantities, or availability of food ingredients associated with the user, including at least stored ingredients, frequently consumed ingredients, and ingredients required for meal preparation.

The term “health information” refers to information indicating a health state of the user, including at least nutritional targets, dietary restrictions, medical conditions, biometric measurements, and other data relevant to determining suitable meals for the user.

The term “preference information” refers to information indicating preferences of the user, including at least favored or disliked foods, taste preferences, cooking style preferences, and lifestyle preferences that influence the selection of meals or menu plans.

The term “behavior history information” refers to information indicating past behavior of the user, including at least purchase history, order history, browsing history, and interaction history with the system or external services.

The term “position information” refers to information indicating a geographical position of the user, including at least location coordinates, region identifiers, and related temporal information used for determining supply locations, provision locations, or route information.

The term “emotion information” refers to information indicating an emotion state of the user, including at least data derived from text analysis, sensor data, explicit user input, or other sources, representing emotions such as stress, joy, sadness, or fatigue.

The term “structured data” refers to data obtained by integrating and organizing heterogeneous user-related information into a predefined format or schema suitable for analysis by the system, including at least tabular data, key-value pairs, or records with defined fields.

The term “ingredient consumption tendency” refers to a tendency or pattern of ingredient usage by the user, derived from behavior history information, and indicating at least frequency, recency, or quantity of consumption of specific ingredients.

The term “health state” refers to a state of the user related to health, derived from health information and optionally behavior history information, and indicating at least current or target nutritional balance, dietary constraints, or other health-related conditions.

The term “budget state” refers to a state of the user related to monetary constraints, derived from user-related information, and indicating at least available budget, spending patterns, and cost preferences for meals or purchases.

The term “emotion state” refers to a state representing a dominant or relevant emotion of the user at a given time, derived from emotion information, and used as a parameter in analysis and prompt generation.

The term “external provision information” refers to information acquired from external sources other than the user and the user terminal, including at least sales promotion information, discount information, and distribution information relevant to products, services, or routes.

The term “sales promotion information” refers to information indicating promotional activities related to products or services, including at least campaigns, special offers, bundled deals, or time-limited promotions.

The term “discount information” refers to information indicating a reduction in price for products or services, including at least coupon data, percentage discounts, fixed-amount discounts, and applicable conditions or validity periods.

The term “distribution information” refers to information indicating availability, delivery conditions, or logistical properties of products or services, including at least stock status, delivery areas, delivery times, and distribution constraints.

The term “prompt sentence” refers to a text string described in a natural language, automatically constructed by the system, and specifying conditions and instructions for the generative AI model to generate at least a meal plan, menu plan, purchase route, or purchase target items.

The term “generative AI model” refers to a machine-learned model that, in response to an input such as a prompt sentence, generates new information or content in a natural language or other format, including at least a model based on deep learning architecture.

The term “meal plan” refers to a plan indicating a set of meals for one or more time periods, including at least dish names, ingredient requirements, and optionally nutritional or cost-related properties.

The term “menu plan” refers to a plan indicating a combination of menu items to be provided to the user, including at least dish or menu item selections and associated preparation or acquisition details.

The term “purchase route” refers to a route for purchasing items required by the user, calculated based on position information and supply locations, and including at least an ordered list of locations, distances, or travel instructions.

The term “purchase target items” refers to items to be acquired by the user according to a meal plan or menu plan, including at least specific ingredients, products, or prepared foods designated for purchase.

The term “necessary ingredient information” refers to information indicating ingredients required to realize a meal plan or menu plan, including at least ingredient types, quantities, and associations with specific dishes.

The term “supply location” refers to a location where items required by the user, including ingredients or prepared foods, can be supplied, such as a retail facility, marketplace, or other provider.

The term “provision location” refers to a location from which items or services can be provided or delivered to the user, including at least restaurants, delivery bases, or other service providers.

The term “route information” refers to information used for calculating a travel route, including at least map data, network links, distances, travel times, and constraints such as operating hours or traffic conditions.

The term “delivery option” refers to an option specifying how items are delivered to the user, including at least a selection of a delivery provider, a delivery route, a delivery time, and associated cost conditions.

The term “information acquisition module” refers to a hardware or software component executed by the processor to acquire user-related information from sensing devices, user terminals, or external information services.

The term “analysis module” refers to a hardware or software component executed by the processor to process structured data and to determine at least ingredient consumption tendency, health state, budget state, and emotion state of the user.

The term “prompt generation module” refers to a hardware or software component executed by the processor to construct a prompt sentence in a natural language based on analysis results and external provision information.

The term “generation-result acquisition module” refers to a hardware or software component executed by the processor to convert a prompt sentence into input data for the generative AI model, to obtain an output result from the generative AI model, and to extract at least a candidate of a meal plan or menu plan and necessary ingredient information from the output result.

The term “optimization module” refers to a hardware or software component executed by the processor to adjust a meal plan or menu plan based on necessary ingredient information and external provision information so as to satisfy user conditions and to achieve cost reduction and burden reduction.

The term “route calculation module” refers to a hardware or software component executed by the processor to identify supply locations or provision locations and to compute an optimal purchase route or delivery option based on position information and necessary ingredient information.

The term “providing module” refers to a hardware or software component executed by the processor to transmit plans, ingredient information, routes, and delivery options to the user terminal, to cause display on the user terminal, and to perform order processing or fixation processing based on user selections.

The term “learning module” refers to a hardware or software component executed by the processor to update at least analysis conditions or prompt-generation conditions based on evaluation information and feedback information received from the user terminal.

The term “evaluation information” refers to information indicating an evaluation by the user of a provided plan, route, or option, including at least rating values, satisfaction indicators, or selection records.

The term “feedback information” refers to information provided by the user that comments on or modifies a provided plan, including at least textual comments, correction requests, or indications of preference changes.

In one embodiment, a server implements the claimed system as a network-connected computing apparatus including at least one processor, a main memory, a non-volatile storage device, a network interface, and an interface to external sensing devices. The server executes a program stored on the non-volatile storage device and loaded into the main memory. The program is implemented, for example, using an operating system such as a general-purpose server operating system, a relational database management system, an in-memory data store, and an application framework. The server communicates with at least one user terminal and at least one sensing device via a communication network using a communication protocol such as HTTPS over TCP/IP.

In one embodiment, the server uses a database system such as a relational database to store structured user-related information and uses an in-memory data store to cache frequently accessed analysis results. The server uses a data processing environment such as a numerical computation library to implement the analysis module, and a deep-learning framework to implement or interface with the generative AI model. The server also uses a routing engine or a mapping service API to compute purchase routes or delivery options based on position information.

In one embodiment, the server acquires user-related information by cooperating with sensing devices and user terminals. A sensing device includes, for example, a smart refrigerator, a body-weight scale, a wearable device, or a point-of-sale system. Such a sensing device transmits ingredient information, health information, or behavior history information in the form of sensor readings or logs to the server via the network. The server stores these data in the database using predefined tables for ingredients, biometric data, and transaction logs. The server acquires preference information and emotion information from the user terminal. The user terminal is, for example, a smartphone, a tablet, or a personal computer executing a client application or a web application. The user uses the user terminal to input explicit preferences, dietary constraints, budget information, and subjective evaluations such as “likes spicy food,” “wants low-sodium meals,” or “prefers short preparation time.” The user also inputs free-text messages such as “I feel stressed today” or “I am in a good mood and want to celebrate.”

In one embodiment, the server uses a text-analysis engine, implemented for example via a natural language processing library or an external sentiment analysis service, to derive emotion information and emotion state from the free-text messages. The server tokenizes text, normalizes tokens, computes embeddings or sentiment scores, and maps such scores to discrete labels such as “stress,” “joy,” or “fatigue.” The server stores these labels and associated confidence values as emotion information linked to the user. The server thereby constructs and maintains a multi-dimensional user profile including ingredient information, health information, preference information, behavior history information, position information, and emotion information.

In one embodiment, the server integrates heterogeneous user-related information into structured data using a predefined schema. The server converts raw sensor logs, transaction records, and user inputs into normalized tables or record structures, for example, a user table, an ingredient table, a health-state table, and a transaction-history table. For each user, the server computes derived features such as ingredient consumption tendency (e.g., purchase frequency and recency per ingredient category), health state (e.g., average daily calorie intake, deviation from target intake), budget state (e.g., average spending per day, remaining budget for a given period), and emotion state (e.g., dominant emotion over a predefined time window). The server stores these derived features as part of the structured data. By aggregating and normalizing all these aspects, the server reduces data redundancy, improves cache locality, and enables faster computation in subsequent analysis and optimization steps.

In one embodiment, the server analyzes the structured data using a set of deterministic algorithms and learned models. The server uses feature extraction routines implemented in a numerical library to compute time-series statistics and category-level consumption metrics from transaction data. The server uses classification or regression models, built for example using a neural network framework, to infer health state or risk scores based on biometric and ingredient consumption data. The server uses an emotion classifier to map text-derived features to emotion labels. The server thereby obtains, for each user, an internal representation of current and predicted ingredient consumption tendency, health state, budget state, and emotion state.

In one embodiment, the server uses external provision information to further refine the analysis. External provision information includes, for example, sales promotion information, discount information, and distribution information provided by retailer systems, online marketplaces, and logistics providers. The server retrieves such information via web APIs or batch imports and normalizes it into tables indicating product identifiers, discount rates, validity periods, store or provider identifiers, stock levels, delivery areas, and expected delivery times. The server joins external provision information with the user's structured data to identify applicable discounts and feasible supply or provision locations.

In one embodiment, the server generates a prompt sentence using a prompt generation module. The server programmatically constructs the prompt sentence as a natural-language text that encodes the analyzed user state and relevant external provision information in a concise and machine-readable manner. Compared to ad-hoc manual prompts, this machine-constructed prompt sentence follows a defined template and uses explicitly named sections (e.g., “User profile,” “Health constraints,” “Budget constraints,” “Emotion state,” “Available promotions,” “Location context,” and “Task specification”). For example, the server generates a prompt sentence such as:
“User's past order history: pizza, pasta, salad (pizza ordered 8 times in the last 30 days). Current promotion: pizza 20% off at a nearby store. User is on a diet, wants a low-calorie dinner within 800 yen, and is currently feeling stressed. The user is near an urban area. Please generate a detailed dinner meal plan for tonight, including dish names, approximate calories, and a short explanation of why each dish matches the user's mood and budget.”

In another example, the server generates a prompt sentence such as:
“User's current location is a district in a city. User is on a diet, budget is 1,000 yen, and emotion is stressed. Please propose the best food delivery options that are low-calorie, budget-friendly, and stress-relieving.”

In another example, the server generates a prompt sentence such as:
“Based on the following receipt and coupon data, and the user's wish to save food costs this week, generate a 3-day meal plan that maximizes coupon usage while keeping each dinner under 700 yen.”

In yet another example, the server generates a prompt sentence such as:
“User's ingredients: chicken breast, tomatoes, lettuce. User's health goal: reduce sodium intake and total calories. User's budget per dinner: 600 yen. User is feeling tired. Please generate two dinner menus using the listed ingredients, minimizing additional purchases, and explain how each menu supports the health goal and emotion state.”

In one embodiment, the server uses a generative AI model implemented as a neural network architecture, such as a multi-layer transformer network having an encoder-decoder or decoder-only structure. The generative AI model is trained on large-scale text data and optionally fine-tuned on domain-specific meal planning and nutrition texts. The model parameters include millions or billions of numerical weights in matrix form, and the model operates with a vocabulary embedding, position encoding, multi-head self-attention layers, feed-forward layers, and normalization layers. The server uses a training regimen involving a loss function such as cross-entropy loss on token prediction, backpropagation for computing gradients, and an optimization algorithm such as stochastic gradient descent with momentum or an adaptive method. During training or fine-tuning, the server can apply data augmentation techniques, such as paraphrasing of recipe texts, random masking of ingredient names, or permutation of meal order, to improve robustness.

In one embodiment, the server converts the prompt sentence into a sequence of tokens via a tokenizer associated with the generative AI model. The server passes the token sequence to the generative AI model's inference engine with parameters such as maximum output length, temperature, and top-k or top-p sampling thresholds. The model generates a sequence of output tokens corresponding to a natural-language response. The server decodes the tokens into text and then parses the text using pattern-matching rules and structured-output conventions enforced by the prompt sentence. For example, the server instructs the model in the prompt sentence to label dishes with numerals and to include calorie estimates and ingredient lists, enabling deterministic extraction of dish names, calories, and ingredients.

In one embodiment, the server uses a generation-result acquisition module to extract from the generated text a candidate meal plan or menu plan and corresponding necessary ingredient information. The server splits the response into lines or segments, detects section headers such as “Dish 1:” or “Shopping list:”, and uses rule-based or machine-learning-based parsers to identify ingredients, quantities, and preparation notes. The server stores these extracted elements as structured records, for example, a list of dishes associated with ingredient requirements, estimated calories, and annotated tags for emotion alignment and budget alignment.

In one embodiment, the server uses an optimization module to adjust the meal plan or menu plan in view of external provision information and internal constraints. The optimization module executes algorithms such as integer linear programming, dynamic programming, or greedy approximation to choose items and sources that minimize total cost subject to budget, health, and emotion constraints. For example, the server defines an objective function that combines cost, distance, and expected satisfaction score, and defines constraints such as total calorie bounds, maximum sodium per meal, and minimum number of servings. The server uses the extracted necessary ingredient information to generate candidate item lists and uses the external provision information to attach prices, discounts, and supply locations. The server then computes an optimal or near-optimal combination of items and sources and modifies the plan accordingly, for example, by substituting ingredients that are discounted or available at closer supply locations.

In one embodiment, the server uses a route calculation module to compute purchase routes or delivery options. The server calls a routing API or executes an internal shortest-path algorithm on a road network graph. The server uses position information from the user terminal and location information from supply locations and provision locations to build a weighted graph, where nodes represent candidate locations and edges represent possible travel paths with weights such as travel time, distance, or cost. The server computes optimal paths, for example using Dijkstra's algorithm or an A* search algorithm with heuristics based on estimated travel time. The server also incorporates distribution information such as store opening hours and delivery time windows to eliminate infeasible paths. The server outputs one or more recommended routes or delivery options, each with associated metrics such as total distance, estimated travel time, and total expected cost.

In one embodiment, the server uses a providing module to generate response messages for the user terminal. The server constructs a data structure that includes a textual description of the meal plan or menu plan, a shopping list with quantities and suggested stores, a representation of the purchase route (e.g., an ordered list of coordinates and turn-by-turn instructions), and a list of delivery options with provider names, menu items, prices, and estimated delivery times. The server transmits this structure to the user terminal via a network API. The user terminal renders the received information on a display, for example by drawing a map with the route overlaid and by listing dishes and prices in a graphical user interface. The user can select one of the suggested plans or options via touch interaction or other input means.

In one embodiment, the user confirms an order or a route selection on the user terminal. The user terminal sends a confirmation message back to the server. The server, in the case of a delivery option, transmits an order request to an external delivery provider's interface, specifying the selected items, delivery address, and payment method. The server receives order confirmations and updates status information, which the user terminal displays as notifications. In the case of an in-store route, the server may provide step-by-step navigation instructions or reminders via the user terminal. By integrating route calculation and plan optimization with generative AI output, the server reduces the number of separate network calls and eliminates redundant recomputation, thereby lowering communication load and improving response time.

In one embodiment, the server uses a learning module to update analysis conditions and prompt-generation conditions based on evaluation information and feedback information. The user uses the user terminal to rate the proposed meal plan, for example on a numerical scale, and to provide free-text feedback such as “too expensive,” “portions too small,” or “not enough vegetables.” The server parses feedback, maps feedback terms onto preference dimensions, and updates the user profile accordingly. The server modifies weight parameters used in the analysis module (for example, giving higher importance to cost sensitivity or portion size) and changes the template or content of future prompt sentences (for example, adding explicit statements such as “avoid small portions” or “prioritize low-cost per calorie”). The server also uses aggregated feedback across multiple users to adjust default weights in optimization algorithms and hyperparameters used for the generative AI model's inference configuration.

In one embodiment, the server improves computer technology itself by structuring the interaction with the generative AI model in a way that reduces non-deterministic behavior and computational waste. By constructing prompt sentences from structured data with explicit sections and constraints, the server narrows the model's output domain and reduces the need for repeated trial-and-error calls. The server thereby reduces the number of inference calls and the average inference time per user session, which improves throughput and reduces computation cost. The structured prompt design also makes parsing of the model's output easier and more reliable, reducing post-processing errors and avoiding manual correction or re-parsing steps.

In one embodiment, the server improves data management by storing all user-related information and derived features in normalized schemas and by caching frequently used representations. This structure avoids repeated parsing of raw logs and repeated feature computation, thereby reducing CPU time and memory usage. When the server receives a new request from the user terminal, the server can re-use existing feature vectors and only update incremental changes (for example, new purchases since the last session). This incremental update strategy reduces latency and improves responsiveness.

In one embodiment, the server improves calculation efficiency for route computation by pruning the search space using user constraints and external provision information before invoking the routing algorithm. For example, the server eliminates supply locations that do not stock required ingredients or that have expired promotions. This pruning reduces the number of nodes and edges in the route graph, thereby reducing the number of operations performed by the routing algorithm and leading to faster computation of optimal paths.

In one embodiment, the generative AI model processes input features and generates outputs based on rules and learned associations that are not equivalent to human manual reasoning. The model uses high-dimensional embeddings and multi-layer attention patterns to combine ingredient preferences, promotions, health constraints, and emotion states in a way that a human would not normally consider in a single step. The server enforces specific, non-conventional prompting patterns, such as including meta-instructions for output structure and constraint handling, which cause the model to follow particular behavioral patterns. These patterns, combined with the optimization and routing modules, result in a composite algorithm that leverages capabilities of both neural inference and deterministic search, rather than merely automating a human decision sequence.

In one embodiment, the learning module updates not only user-specific parameters but also global model-invocation strategies, such as dynamic adjustment of sampling parameters or the decision to use a smaller or larger model variant depending on the complexity of the task. The server thereby manages computational resources adaptively, allocating more computation to high-impact decisions and less to trivial or repetitive scenarios. This results in an overall reduction of system-wide computation load while maintaining or improving recommendation quality.

In another embodiment, the server uses an alternative generative AI model, such as a sequence-to-sequence network or a mixture-of-experts model, provided that the model is capable of ingesting a prompt sentence and outputting natural-language or structured descriptions of meal plans and related options. The server can deploy such models on specialized hardware accelerators, such as graphics processing units or tensor processing units, to further improve inference speed. The server can also vary the architecture of the analysis module, for example by using gradient-boosted trees or probabilistic graphical models for certain sub-tasks instead of neural networks, as long as the system continues to generate structured data and prompt sentences as described.

In another embodiment, the user terminal takes different forms, including a wearable device with a small display or a voice-controlled smart device. In such cases, the server adapts the presentation content to the capabilities of the device while preserving the underlying plan structures and route recommendations. The user can interact through voice commands, and the server interprets voice input via a speech recognition module before feeding it into the analysis and prompt generation pipeline.

In yet another embodiment, the server obtains distribution information from different types of providers, such as grocery stores, restaurants, meal-kit services, or community-supported agriculture groups. The server unifies such information into a common schema, allowing the optimization module and route calculation module to treat all sources uniformly. This unification enables the server to identify hybrid options, such as combining in-store purchases with delivery of certain items, thereby achieving better cost and time trade-offs.

Through these embodiments, the server, the terminal, and the generative AI model cooperate to implement a concrete, technically grounded processing pipeline. The pipeline improves the functioning of the computer system itself by reducing redundant computations, managing data more effectively, constraining generative model behavior for more reliable outputs, and optimizing routing and promotion application in a single integrated flow.

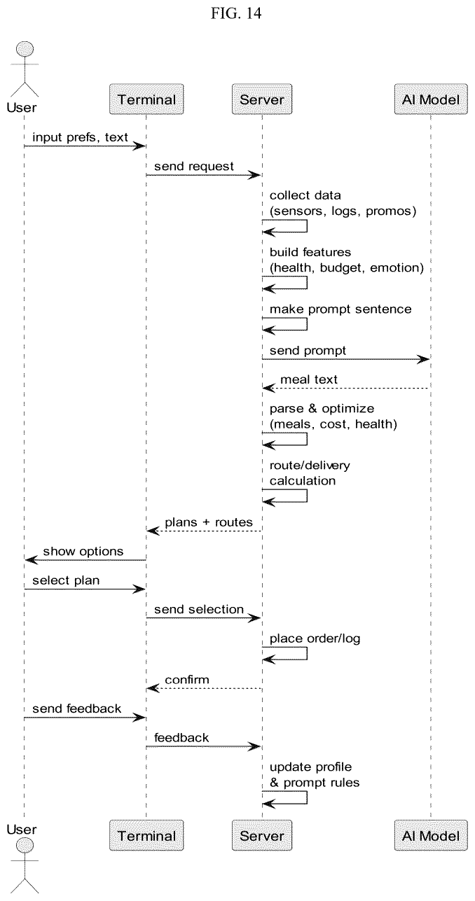

The following describes the processing flow using FIG. 14.

### Step 1:

Server acquires user-related raw data from sensing devices and external services.

Server receives, as input, sensor readings, transaction logs, and external provision information from at least one sensing device and at least one external information service. This input includes ingredient states (e.g., “refrigerator inventory: chicken, tomato, lettuce”), health measurements (e.g., weight, step count), purchase history (e.g., item IDs, timestamps, prices), and promotion data (e.g., coupons, discounts, stock status). Server parses incoming messages, validates formats, and stores the raw records into database tables such as “sensor_log,” “purchase_log,” and “promotion_raw.” As output, server produces normalized raw data records indexed by user identifier and timestamp.

### Step 2:

Server acquires preference information, budget information, and free-text requests from the user terminal.

User enters, as input, explicit preferences and conditions on the terminal, such as “low-sodium meals,” “budget 800 yen per dinner,” and free-text comments like “I feel stressed today and want something easy.” Terminal bundles these texts and numeric values into a request message and sends it to server. Server receives this message, extracts fields such as preference flags, budget limits, and raw user text, and stores them in tables such as “user_preference” and “session_request.” As output, server generates structured preference and budget records associated with the current session.

### Step 3:

Server derives emotion information and emotion state from user text.

Server takes, as input, the free-text messages and comments stored in “session_request.” Server applies a text-processing pipeline: tokenization, normalization, and, optionally, vectorization into embeddings. Server then executes an emotion classifier (for example, a neural network or external sentiment API) to compute scores for emotion categories such as stress, joy, sadness, and fatigue. Based on these scores, server determines a primary emotion label (e.g., “stressed”) and an intensity value. Server writes these values into an “emotion_state” table. As output, server produces an emotion state record referencing the user and the current session.

### Step 4:

Server integrates heterogeneous user-related information into structured feature data.

Server receives, as input, raw sensor data, purchase logs, promotion data, preference records, and emotion state records from the previous steps. Server performs data processing operations including joins on user ID and timestamp ranges, unit normalization (e.g., grams vs. pieces), category mapping (e.g., mapping product IDs to ingredient categories), and time-window aggregation (e.g., “count of poultry purchases in last 30 days”). Server calculates derived features such as ingredient consumption tendency vectors, recent daily calorie estimates, remaining budget for the week, and a summarized emotion profile. Server stores the results in a “user_feature” table or in-memory feature store. As output, server generates a structured feature set representing the current state of the user.

### Step 5:

Server evaluates health state and budget state from feature data.

Server takes, as input, the feature set from “user_feature” for the current user, including consumption tendencies and health-related metrics. Server compares the computed nutritional intake to target values (e.g., recommended daily calories, sodium limits) and classifies the health state (e.g., “slightly above target calories,” “sodium too high”). Server also compares recent spending to budget settings to classify the budget state (e.g., “under budget,” “near budget limit”). Server writes these classifications and numeric margins into a “user_state” table. As output, server produces a comprehensive state record containing health state and budget state attributes.

### Step 6:

Server selects applicable promotions and supply/provision locations.

Server receives, as input, the user's feature set and state record, along with normalized promotion and distribution data. Server filters promotions by product categories that match the user's consumption tendency or necessary ingredients inferred from preferences (e.g., “user often eats poultry; chicken is 30% off”). Server then filters supply and provision locations based on distribution constraints such as user region and delivery coverage. Server records applicable promotions and feasible supply/provision locations in tables such as “applicable_promotion” and “feasible_location.” As output, server produces a set of promotion entries and locations relevant to the user.

### Step 7:

Server constructs a structured prompt sentence for the generative AI model.

Server takes, as input, the user state (health, budget, emotion), feature set (consumption tendencies), applicable promotions, feasible locations, and the explicit user request text. Server formats this information into a natural-language prompt sentence according to a predefined template with labeled sections. Server includes, for example, past orders, current promotions, current emotion, budget limits, and the requested planning horizon. Server may generate a prompt sentence such as:
“User's past order history: pizza, pasta, salad (pizza ordered 8 times in the last 30 days). Current promotion: pizza 20% off at a nearby store. User is on a diet, wants a low-calorie dinner within 800 yen, and is currently feeling stressed. The user is near an urban area. Please generate a detailed dinner meal plan for tonight, including dish names, approximate calories, and a short explanation of why each dish matches the user's mood and budget.”

Server stores the generated prompt sentence in a “prompt_log” table. As output, server provides a finalized prompt sentence string ready for inference.

### Step 8:

Server executes inference of the generative AI model using the prompt sentence.

Server receives, as input, the prompt sentence string from “prompt_log.” Server tokenizes the prompt sentence into tokens according to the vocabulary of the generative AI model and builds an input tensor. Server sends this tensor, along with inference parameters (e.g., maximum token count, temperature, top-k threshold), to a generative AI model instance hosted on a model-serving engine. The generative AI model processes the tokens through its neural network layers and returns an output token sequence. Server decodes the tokens back into text. As output, server obtains a natural-language response that describes candidate meal plans, dish lists, or menu options.

### Step 9:

Server parses the generative AI model's response into structured candidate plans.

Server takes, as input, the generated text output from the generative AI model. Server applies parsing rules, such as splitting by numbered list entries, detecting headings like “Dish 1,” and scanning for patterns like “Calories:” or “Ingredients:”. Server extracts dish names, approximate calorie values, and ingredient lists. Server converts ingredient names into standardized identifiers via a mapping table. Server stores dish entries and required ingredient entries into tables such as “candidate_meal_plan” and “candidate_ingredient.” As output, server produces a structured set of candidate meal plans associated with detailed ingredient requirements.

### Step 10:

Server optimizes candidate meal plans using promotions, budget, and health constraints.

Server receives, as input, the structured candidate plans, applicable promotions, user budget state, and health state. Server computes, for each candidate plan, the estimated cost by summing ingredient prices and subtracting applicable discounts. Server also computes nutritional metrics (e.g., total calories, sodium) for each plan. Using these metrics, server executes an optimization algorithm to select or modify plans. For example, server may solve a constrained cost-minimization problem where each plan must not exceed the budget per meal and must keep calories below a threshold. Server may also substitute ingredients with discounted alternatives while respecting health constraints. Server writes the optimized plans into an “optimized_meal_plan” table. As output, server produces one or more optimized meal plans with associated cost and health annotations.

### Step 11:

Server computes optimal purchase routes and delivery options.

Server takes, as input, the optimized meal plans, required ingredients, feasible supply/provision locations, and position information for the user. Server constructs a graph where nodes represent candidate stores or providers and edges represent travel paths with associated distances and times. Server evaluates which locations carry required ingredients or prepared dishes and prunes locations that do not meet stock or delivery constraints. Server then runs a routing algorithm on the pruned graph to compute an optimal route (e.g., minimal travel time) covering all required items. For delivery options, server queries provider APIs to obtain menu availability, fees, and delivery times and assembles viable combinations that fulfill the ingredient or dish requirements. Server stores route descriptions and delivery options in a “route_option” table. As output, server generates a set of recommended purchase routes and delivery options with cost and time metrics.

### Step 12:

Server assembles a final recommendation package and sends it to the user terminal.

Server receives, as input, the optimized meal plans, the computed routes, and the delivery options. Server builds a response object that includes: textual descriptions of meal plans, per-dish calorie and cost information, shopping lists with item quantities and suggested stores, route summaries, and a list of delivery alternatives with estimated delivery times. Server serializes this response into a message and transmits it to the terminal over the network. As output, server provides a complete recommendation package that the terminal can render.

### Step 13:

Terminal displays recommendations and accepts user selection.

Terminal takes, as input, the recommendation package from the server. Terminal parses the received structures and renders them on the display, for example listing dishes with calories and savings, drawing a route map, and showing delivery choices with “Order” buttons. User reviews the displayed options and selects a desired plan, route, or delivery option using touch or other input means. Terminal collects the selection (e.g., “Plan A with delivery option 2”) and sends a selection message back to the server. As output, terminal produces a user selection record transmitted to the server.

### Step 14:

Server executes order or plan confirmation and logs the session.

Server receives, as input, the user selection record from the terminal. For a delivery selection, server composes an order request including selected dishes, address, and payment metadata, and transmits it to the corresponding provider interface. For an in-store route selection, server records the chosen route and may generate navigation hints or reminders. Server logs all finalized decisions, including selected plan, applied promotions, route or delivery choice, and timestamps, into persistent storage such as “session_log.” As output, server generates confirmation data (e.g., “order confirmed, expected delivery 18:30” or “route confirmed”) and updates persistent logs for future analysis.

### Step 15:

Server collects user evaluation and feedback for continuous learning.

User, after consuming the meal or following the plan, enters, as input, ratings and comments on the terminal, such as “4 out of 5,” “too salty,” or “portion size too small.” Terminal transmits these evaluation and feedback messages to the server. Server parses the feedback, recognizes key terms and numeric ratings, and updates the user profile and learning parameters. Server adjusts weights used in the analysis (e.g., increasing importance of sodium limit), updates flags in preference records (e.g., “prefers larger portions”), and modifies templates for future prompt sentences (e.g., adding “avoid salty food” or “ensure sufficient portion size”). As output, server produces updated user-specific parameters and prompt-generation conditions, which influence subsequent executions of the generative AI model and optimization routines.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

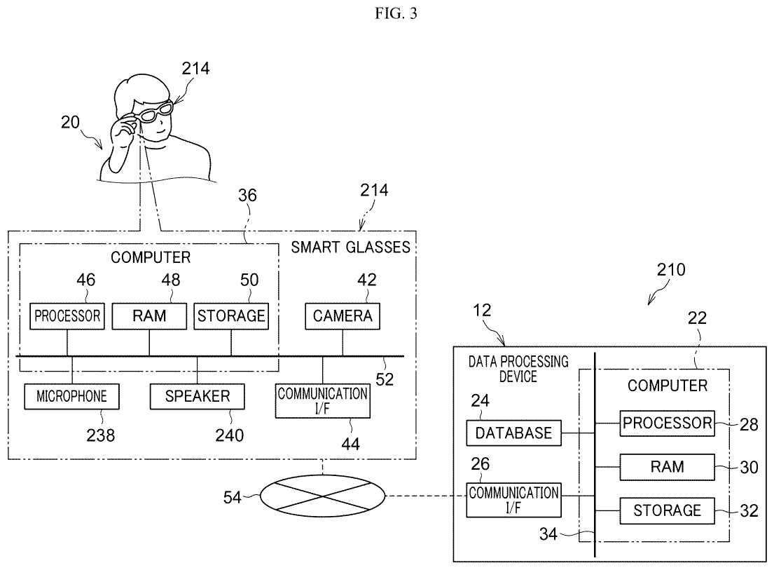

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

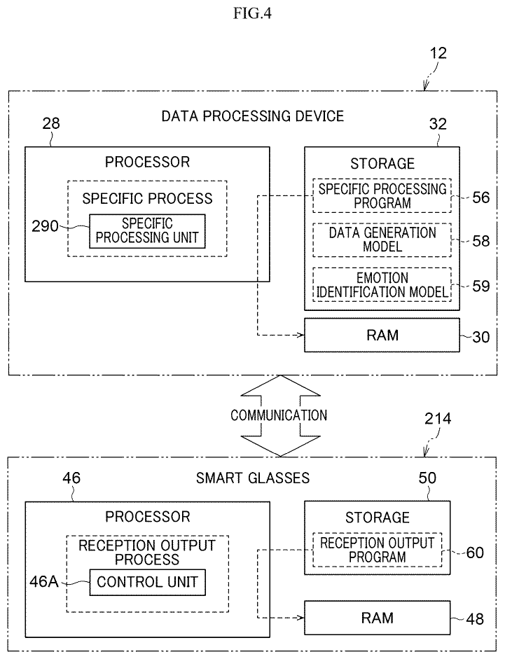

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

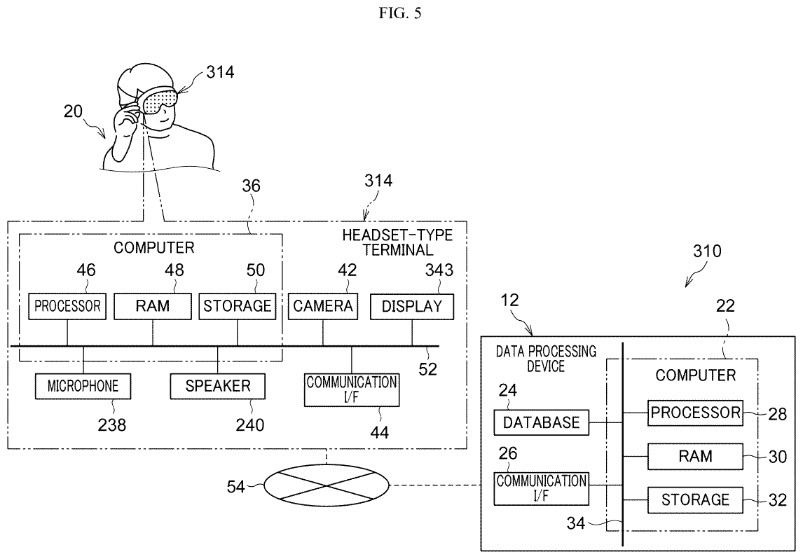

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

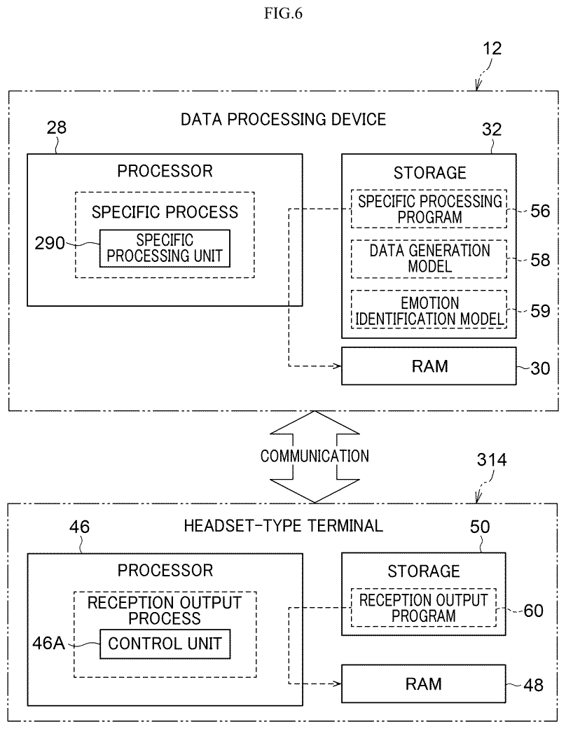

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

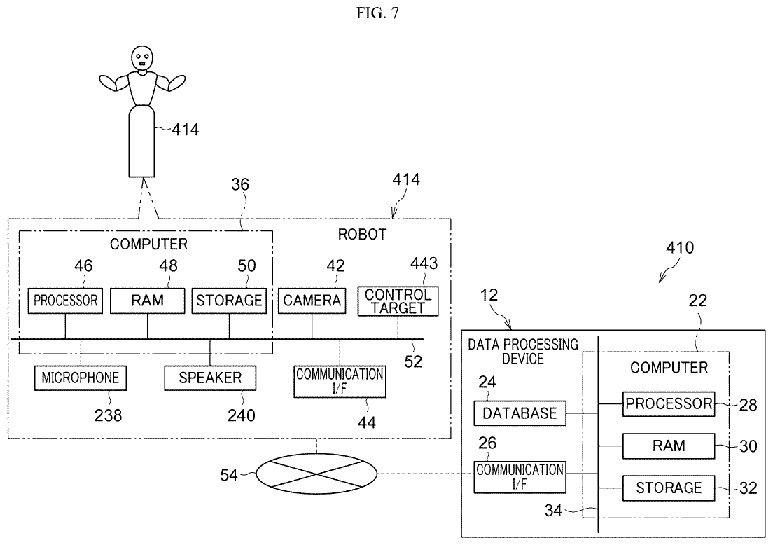

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

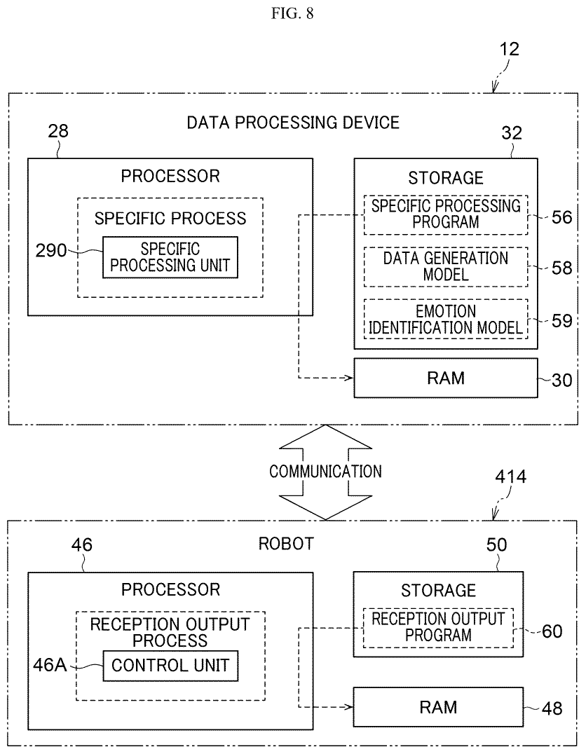

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

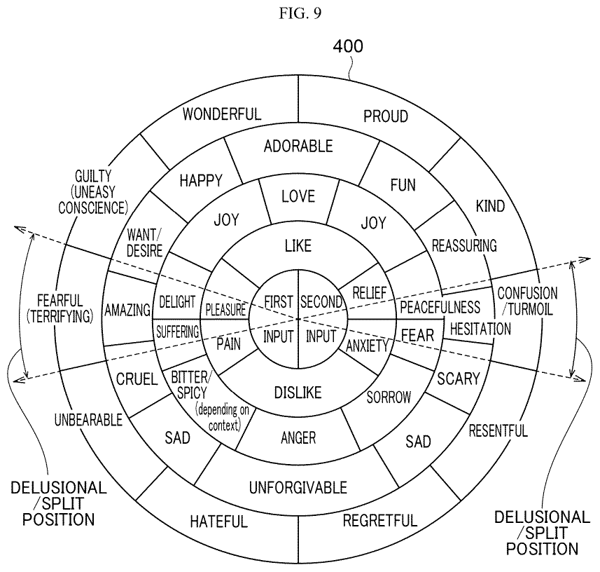

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

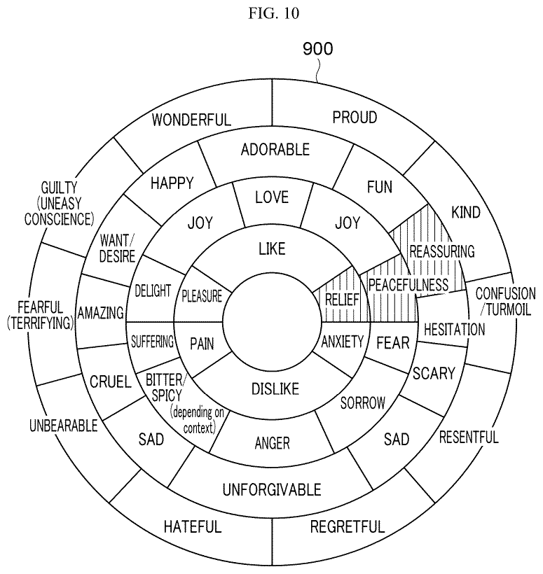

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
automatically acquire, from an application having a communication processing function, message data in a natural language input by a user,
perform, on text information included in the message data, a text analysis process using a natural language processing program, the text analysis process including morphological analysis, part-of-speech tagging, phrase segmentation, semantic analysis, and entity extraction, and generate structured data including parameters indicating a user intent, a time range, a category, and transaction-related information,
store, based on the structured data, a data group including purchase history data of the user and coupon data in an information storage device, or acquire the data group from the information storage device, by using a relational database management component, and manage the data group in association with each user,
select or aggregate, based on the user intent obtained by the text analysis process and the structured data, the purchase history data and the coupon data acquired from the relational database management component, and generate machine-readable information including a result of the selection or aggregation,
generate, based on the machine-readable information and the message data in the natural language from the user, a prompt sentence to be input to a generative AI model, input the prompt sentence to the generative AI model, and cause the generative AI model to generate a response message in the natural language including menu information and an explanatory text corresponding to the menu information, and
transmit the response message in the natural language to a terminal of the user as response data for the application having the communication processing function.

### Supplementary 2

The system according to supplementary 1,
wherein the processor is configured to, when the message data in the natural language is a registration request for purchase history or received data including transaction-related information, extract, in the text analysis process, entities indicating an item name, a quantity, a price, and a transaction date and time, and store the entities as the purchase history data in the relational database management component, and, when the message data in the natural language is an inquiry request for coupon information, select coupon data within a valid period based on the time range and the category included in the structured data, and include a result of the selection in the prompt sentence to be input to the generative AI model.

### Supplementary 3

The system according to supplementary 1,
wherein the processor is configured to, after obtaining the response message including the menu information and the explanatory text from the generative AI model, optimize the menu information based on the purchase history data and the coupon data for each user, and transmit, as response data for presentation to the terminal of the user via the application having the communication processing function, the optimized menu information and coupon information associated with the optimized menu information.

### Application Example 1

### Supplementary 1

A system comprising a processor,
wherein the processor is configured to
acquire, via a communication interface and an external connection mechanism, information assets including transaction record information, discount information, and advertisement information from a communication application for communication between users, automatically convert the acquired information assets into structured data, and store the structured data in a storage device,
receive current location information of a user from a location acquisition mechanism, identify a sales point within a predetermined range based on the current location information, and extract, from the storage device, discount information and advertisement information associated with the sales point,
input the transaction record information and purchase history information of the user into an analysis processing mechanism, perform statistical processing and machine learning processing to generate feature values representing a purchase tendency of the user, and calculate candidate items and candidate meal plans,
generate, based on context information including the candidate meal plans, the candidate items, the current location information, and the discount information, a prompt sentence that instructs execution of a generative AI model operating on a generation processing device,
input the prompt sentence into the generative AI model, cause the generative AI model to generate natural language text including a recommendation message for the user according to the context information, associate the candidate meal plans and the candidate items with the natural language text, and transmit the natural language text and the association to an information processing terminal of the user,
calculate a movement route within the sales point or between a plurality of sales points based on the candidate meal plans and the candidate items, and transmit movement route information to the information processing terminal of the user, and
receive operation history information of the user and new transaction record information transmitted from the information processing terminal of the user, and update the purchase history information and the feature values to iteratively improve calculation processing of the candidate meal plans and the candidate items and generation processing of the prompt sentence.

### Supplementary 2

The system according to supplementary 1,
wherein the processor is configured to
convert a plurality of types of structured data, including analyzed purchase tendency information, sales point information based on the current location, available discount information and advertisement information, and calculated movement route information, into text format when generating the prompt sentence to be input to the generative AI model, embed the converted structured data into the prompt sentence as an integrated instruction statement, and cause the generative AI model to generate a recommendation message that integrally includes a meal plan proposal, a product proposal, and movement route guidance for the user.

### Supplementary 3

The system according to supplementary 1,
wherein the processor is configured to
use the analysis processing mechanism to calculate an evaluation index for each user based on the purchase history information, the discount information, and the advertisement information, rank the candidate meal plans and the candidate items according to the evaluation index, and include a result of the ranking in the prompt sentence to be input to the generative AI model so as to optimize the meal plans and product candidates presented to the user.

### Example 2

### Supplementary 1

A system comprising a processor,
wherein the processor is configured to
receive request information including user identification information from a user terminal, and, based on the request information, access an information storage device to retrieve purchase history information and price discount information associated with the user, and obtain the purchase history information and the price discount information for the user,
aggregate and normalize the obtained purchase history information and price discount information by execution of a data processing program to extract frequent item information and applicable discount information and to analyze a food ingredient consumption tendency and a price discount usage tendency of the user,
generate, in a natural language, a prompt sentence including at least the frequent item information and the applicable discount information, the prompt sentence further specifying an output format and menu generation conditions, based on an analysis result and condition designation information received from the user,
input the generated prompt sentence into a generative artificial intelligence model and obtain menu candidate information output from the generative artificial intelligence model,
associate each meal component included in the obtained menu candidate information with the purchase history information and the price discount information, calculate price information and discount application information for the meal component, evaluate and rank the menu candidate information based on economic efficiency and ingredient utilization efficiency for the user, and generate optimized menu information, and
transmit the generated optimized menu information to the user terminal.

### Supplementary 2

The system according to supplementary 1,
wherein the processor is configured to
generate the prompt sentence by explicitly describing, in the prompt sentence, constraint information including at least a menu planning period, a meal category, nutritional conditions, cooking time conditions, and output format conditions in addition to the frequent item information and the applicable discount information included in the analysis result, and cause the generative artificial intelligence model to generate the menu candidate information including, for each day, a menu name, used ingredients, cooking procedures, and a structured data format.

### Supplementary 3

The system according to supplementary 1,
wherein the processor is configured to
normalize ingredient names included in the menu candidate information obtained from the generative artificial intelligence model, based on standardized ingredient information stored in the information storage device, associate the normalized ingredient information with the purchase history information and the price discount information to identify ingredients that the user is likely to already possess and ingredients to which a price discount is applicable, and generate the optimized menu information by calculating and comparatively evaluating an estimated cost and a discount application effect for each menu candidate based on an identification result.

### Application Example 2

### Supplementary 1

A system comprising a processor,
wherein the processor is configured to
acquire, by an acquisition unit, user-related information including ingredient information, health information, preference information, behavior history information, position information, and emotion information, from at least one sensing device, user terminal, or external information service,
integrate and organize the acquired user-related information as structured data and, based on the structured data, analyze, by an analysis unit, at least a consumption tendency of ingredients of the user, a health state of the user, a budget state of the user, and an emotion state of the user,
generate, by a prompt generation unit, a prompt sentence described in a natural language, the prompt sentence specifying, based on a result of the analysis and on external provision information including at least sales promotion information, discount information, and distribution information, at least one of a meal plan or menu plan, a purchase route, and purchase target items to be provided to the user,
convert, by a generation-result acquisition unit, the prompt sentence generated by the prompt generation unit into input data for a generative AI model, input the input data to the generative AI model, and extract, from an output result of the generative AI model, at least a candidate of a meal plan or menu plan and necessary ingredient information corresponding to the candidate,
optimize, by an optimization unit, the meal plan or menu plan, based on the necessary ingredient information extracted by the generation-result acquisition unit and the sales promotion information and the discount information, such that the meal plan or menu plan satisfies at least a budget condition and a health condition of the user while achieving cost reduction and burden reduction for the user,
calculate, by a route calculation unit, based on the position information and the necessary ingredient information obtained by the optimization unit, at least one of available supply locations or provision locations, and, using route information, compute an optimal purchase route or delivery option,
provide, by a providing unit, the meal plan or menu plan, the necessary ingredient information, the purchase route, and the delivery option obtained by the optimization unit and the route calculation unit to the user terminal, cause display of these on the user terminal, and execute, based on a selection operation on the user terminal, at least one of order processing or fixation processing of an execution plan, and
update, by a learning unit, at least one of analysis conditions used by the analysis unit or generation conditions used by the prompt generation unit, based on evaluation information and feedback information acquired from the user terminal.

### Supplementary 2

The system according to supplementary 1,
wherein the processor is configured to cause the route calculation unit to acquire, based on the position information and the necessary ingredient information, a plurality of the supply locations or the provision locations as candidates, perform computation of data relating to at least route distance, required time, business conditions, and cost conditions to evaluate the purchase route or the delivery option, and determine, based on a result of the evaluation, at least one route or delivery option to be recommended to the user.

### Supplementary 3

The system according to supplementary 1,
wherein the processor is configured to cause the prompt generation unit to generate the prompt sentence by explicitly describing, in the prompt sentence, the emotion state of the user, a preference tendency of the user, and past selection results of the user, based on a user profile including at least the result of the analysis by the analysis unit, the evaluation information, and the feedback information, such that the generative AI model generates, in response to the prompt sentence, at least one of a meal plan or menu plan, an eating-out proposal, and a cost reduction proposal suitable for the user.

What is claimed is:

1. A system comprising:
circuitry configured to
receive, via a packet-switched network, sensor signal data collected by a sensing device associated with a terminal device;
process the sensor signal data using an analytical computation module to generate a set of feature parameters representing at least a temporal consumption pattern and a physiological state classification;
construct, based on the set of feature parameters, a structured input sequence comprising natural language instruction tokens for a generative neural network model;
input the structured input sequence to the generative neural network model and obtain, from the generative neural network model, inference output data comprising a structured set of candidate data records; and
transmit, via the packet-switched network, at least a portion of the inference output data to the terminal device for rendering on a display of the terminal device.

2. The system according to claim 1, wherein the circuitry is further configured to normalize the sensor signal data by mapping raw identifier values to a standardized taxonomy stored in a storage device prior to generating the set of feature parameters.

3. The system according to claim 2, wherein the circuitry is further configured to aggregate the normalized sensor signal data by grouping data entries according to category identifiers and computing frequency metrics over a plurality of configurable time windows to derive the temporal consumption pattern.

4. The system according to claim 3, wherein the sensor signal data comprises data representing types and quantities of food ingredients detected by the sensing device, and wherein the temporal consumption pattern represents a frequency and recency of usage of specified food ingredient categories.

5. The system according to claim 4, wherein the circuitry is further configured to retrieve, from the storage device, purchase history data and discount condition data associated with the terminal device, and to incorporate the purchase history data and the discount condition data into the structured input sequence such that the generative neural network model generates the candidate data records reflecting cost optimization based on the purchase history data and the discount condition data.

6. The system according to claim 1, wherein the circuitry is further configured to receive, via the packet-switched network, an input character sequence from the terminal device, and to perform a text analysis process on the input character sequence using a natural language processing module, the text analysis process comprising at least tokenization, part-of-speech classification, and entity extraction, to generate structured parameter data indicating an intent classification label and one or more extracted entity values.

7. The system according to claim 6, wherein the circuitry is further configured to store, in the storage device using a relational data management component, a data group comprising at least first-type historical transaction data and second-type conditional benefit data in association with an identifier of the terminal device, based on the structured parameter data.

8. The system according to claim 7, wherein the circuitry is further configured to select or aggregate, based on the intent classification label and the structured parameter data, a subset of the first-type historical transaction data and the second-type conditional benefit data, and to incorporate a result of the selection or aggregation as machine-readable context data into the structured input sequence.

9. The system according to claim 8, wherein the first-type historical transaction data comprises purchase history records including item identifiers, quantities, prices, and transaction timestamps, and wherein the second-type conditional benefit data comprises coupon records including applicable item categories and validity period data.

10. The system according to claim 1, wherein the generative neural network model comprises a transformer-based architecture including an embedding layer, a plurality of self-attention layers, a plurality of feed-forward layers, and an output projection layer, and wherein the circuitry is further configured to set at least a sampling temperature parameter and a maximum output token count parameter prior to inputting the structured input sequence.

11. The system according to claim 10, wherein the circuitry is further configured to parse the inference output data by detecting section delimiter tokens and label tokens specified in the structured input sequence, and to extract, from the parsed inference output data, a plurality of structured record entries each comprising at least a name field, an attribute list field, and a procedural instruction field.

12. The system according to claim 11, wherein each structured record entry corresponds to a proposed meal composition, and wherein the attribute list field comprises ingredient identifiers and associated nutritional parameter values.

13. The system according to claim 1, wherein the circuitry is further configured to receive, via the packet-switched network, position coordinate data from the terminal device, and to identify, using a geospatial index structure stored in the storage device, at least one supply location within a predetermined distance from coordinates indicated by the position coordinate data.

14. The system according to claim 13, wherein the circuitry is further configured to compute, using a graph-based path computation algorithm, an optimized traversal sequence through the at least one supply location based on locations of items identified in the inference output data, and to transmit traversal sequence data to the terminal device.

15. The system according to claim 1, wherein the circuitry is further configured to determine an affective state indicator based on at least one of text-derived sentiment data or sensor-derived biometric data associated with the terminal device, and to incorporate the affective state indicator into the structured input sequence.

16. The system according to claim 1, wherein the circuitry is further configured to evaluate and rank the candidate data records based on at least an economic efficiency metric and a resource utilization efficiency metric, and to select a subset of the candidate data records for inclusion in the inference output data transmitted to the terminal device.

17. The system according to claim 1, wherein the circuitry is further configured to receive, via the packet-switched network, operation history data and evaluation feedback data from the terminal device, and to update at least one of analysis parameters used by the analytical computation module or generation parameters used in constructing the structured input sequence based on the operation history data and the evaluation feedback data.

18. A system comprising:
circuitry configured to
receive, via a packet-switched network, sensor signal data from a sensing device and an input character sequence from a terminal device;
perform a text analysis process on the input character sequence comprising tokenization, entity extraction, and intent classification to generate structured parameter data;
process the sensor signal data using an analytical computation module to generate feature parameters representing a temporal consumption pattern and a physiological state classification;
determine an affective state indicator based on at least one of text-derived sentiment data or sensor-derived biometric data;
construct a structured input sequence for a generative neural network model, the structured input sequence incorporating the structured parameter data, the feature parameters, and the affective state indicator;
input the structured input sequence to the generative neural network model and obtain inference output data comprising candidate data records;
evaluate and rank the candidate data records based on an economic efficiency metric derived from historical transaction data and conditional benefit data retrieved from a storage device; and
transmit, via the packet-switched network, ranked inference output data to the terminal device for rendering on a display of the terminal device.

19. The system according to claim 18, wherein the circuitry is further configured to normalize ingredient name data included in the candidate data records by matching the ingredient name data against standardized reference data stored in the storage device, and to associate normalized ingredient identifiers with the historical transaction data and the conditional benefit data to compute, for each candidate data record, an estimated cost value and a discount application value.

20. A method performed by a system comprising circuitry, the method comprising:
receiving, via a packet-switched network, sensor signal data collected by a sensing device associated with a terminal device;
processing the sensor signal data using an analytical computation module to generate a set of feature parameters representing at least a temporal consumption pattern and a physiological state classification;
constructing, based on the set of feature parameters, a structured input sequence comprising natural language instruction tokens for a generative neural network model;
inputting the structured input sequence to the generative neural network model and obtaining, from the generative neural network model, inference output data comprising a structured set of candidate data records; and
transmitting, via the packet-switched network, at least a portion of the inference output data to the terminal device for rendering on a display of the terminal device.
